SEQUENCE LISTING (17 sequences)

SEQ ID NO: 1 - protein, 5 aa, artificial
  CDR1 = hypervariable region 1 of heavy chain of AIN457
NYWMN

SEQ ID NO: 2 - protein, 17 aa, ARTIFICIAL
  CDR2 = hypervariable region 2 of heavy chain of AIN457
AINQDGSEKYYVGSVKG

SEQ ID NO: 3 - protein, 18 aa, ARTIFICIAL
  CDR3 = hypervariable region 3 of heavy chain of AIN457
DYYDILTDYYIHYWYFDL

SEQ ID NO: 4 - protein, 12 aa, ARTIFICIAL
  CDR1′ = hypervariable region 1 of light chain of AIN457
RASQSVSSSYLA

SEQ ID NO: 5 - protein, 7 aa, ARTIFICIAL
  CDR2′ = hypervariable region 2 of light chain AIN457
GASSRAT

SEQ ID NO: 6 - protein, 9 aa, ARTIFICIAL
  CDR3′ = hypervariable region 3 of light chain AIN457
QQYGSSPCT

SEQ ID NO: 7 - DNA, 381 bp, HOMO SAPIENS
  CDS (1)..(381)
gaggtgcagttggtggagtctgggggaggcttggtccagcctggggggtccctgagactctcctgtgcagcctctggattcacctttagtaactattggatgaactgggtccgccaggctccagggaaagggctggagtgggtggccgccataaaccaagatggaagtgagaaatactatgtgggctctgtgaagggccgattcaccatctccagagacaacgccaagaactcactgtatctgcaaatgaacagcctgagagtcgaggacacggctgtgtattactgtgtgagggactattacgatattttgaccgattattacatccactattggtacttcgatctctggggccgtggcaccctggtcactgtctcctca

SEQ ID NO: 8 - protein, 127 aa, HOMO SAPIENS [sequence omitted]

SEQ ID NO: 9 - DNA, 327 bp, HOMO SAPIENS
  CDS (1)..(327)
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtgcaccttcggccaagggacacgactggagattaaacga

SEQ ID NO: 10 - protein, 109 aa, HOMO SAPIENS
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 11 - protein, 10 aa, artificial
  CDR1-x = hypervariable domain x of heavy chain of AIN457
GFTFSNYWMN

SEQ ID NO: 12 - protein, 11 aa, artificial
  CDR2-x = hypervariable domain of heavy chain x of AIN457
AINQDGSEKYY

SEQ ID NO: 13 - protein, 23 aa, ARTIFICIAL
  CDR3-x = hypervariable domain x of heavy chain AIN457
CVRDYYDILTDYYIHYWYFDLWG

SEQ ID NO: 14 - DNA, 711 bp, homo sapiens
  CDS (1)..(708)
accatggaaaccccagcggagcttctcttcctcctgctactctggctcccagataccaccggagaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtgcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 15 - protein, 236 aa, homo sapiens
TMETPAELLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 783 bp, homo sapiens
  CDS (1)..(780)
accatggaattggggctgagctgggttttccttgttgctattttagaaggtgtccactgtgaggtgcagttggtggagtctgggggaggcttggtccagcctggggggtccctgagactctcctgtgcagcctctggattcacctttagtaactattggatgaactgggtccgccaggctccagggaaagggctggagtgggtggccgccataaaccaagatggaagtgagaaatactatgtgggctctgtgaagggccgattcaccatctccagagacaacgccaagaactcactgtatctgcaaatgaacagcctgagagtcgaggacacggctgtgtattactgtgtgagggactattacgatattttgaccgattattacatccactattggtacttcgatctctggggccgtggcaccctggtcactgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccataa

SEQ ID NO: 17 - protein, 260 aa, homo sapiens
TMELGLSWVFLVAILEGVHCEVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCP

(19) United States
(12) Patent Application Publication
(10) Pub. No.: US 2015/0064193 A1
(43) Pub. Date: Mar. 5, 2015

(54) METHODS OF TREATING PSORIATIC ARTHRITIS (PSA) USING IL-17 ANTAGONISTS AND PSA RESPONSE OR NON-RESPONSE ALLELES

(71) Applicant: Ying Wang, Waltham, MA (US)

(72) Inventor: Ying Wang, Waltham, MA (US)

(73) Assignee: Novartis AG, Basel (CH)

(21) Appl. No.: 14/358,504

(22) PCT Filed: Jun. 7, 2012
(86) PCT No.: PCT/US12/41310

§ 371 Date: Aug. 13, 2014

Related U.S. Application Data
(60) Provisional application No. 61/624,564, filed on Apr. 16, 2012.

(30) Foreign Application Priority Data
Nov. 21, 2011 (IQ) 370/2011

(51) Int. Cl. C07K 16/24; A61K 45/06; C12Q 1/68; A61K 39/395

(52) U.S. Cl. CPC C07K 16/244; A61K 39/3955; A61K 45/06; C12Q 1/6883; A61K 2039/505
USPC 424/142.1; 435/6.11

(57) ABSTRACT

The disclosure is directed to predictive methods and personalized therapies for treating psoriatic arthritis (PsA). Specifically, this disclosure relates to methods of treating a patient having PsA by selectively administering an IL-17 antagonist, e.g., an IL-17 antibody, such as secukinumab, to the PsA patient on the basis of that patient being predisposed to have a favorable response to treatment with the IL-17 antagonist. Also disclosed herein are diagnostic methods useful in predicting the likelihood that a patient having PsA will respond to treatment with an IL-17 antagonist, e.g., an IL-17 antibody, such as secukinumab.

### RELATED APPLICATIONS

This application claims priority to Iraq patent application No. 370/2011, filed on Nov. 21, 2011, and U.S. Provisional Patent Application No. 61/624,564, filed on Apr. 16, 2012, both of which are incorporated by reference in their entirety herein.

### TECHNICAL FIELD

The disclosure is directed to novel personalized therapies and methods for use in treating patients having psoriatic arthritis (PsA).

### BACKGROUND OF THE DISCLOSURE

PsA is an immune-mediated chronic inflammatory disease belonging to the spectrum of conditions commonly referred to as spondyloarthritidies (SpA). While SpAs are diverse in their clinical presentation, common environmental and genetic factors are suspected in SpA-afflicted individuals (Turkiewicz and Moreland (2007) Arthritis Rheum 56(4):1051-66; Gladman (2009) Dermatol Ther. 22:40-55). This latter notion was recently corroborated by findings in a large-scale single nucleotide polymorphism (SNP) scan study, where IL23R variants that were previously linked to Crohn's disease and psoriasis (diseases that may both co-exist with spondylarthritides) conferred risk to developing ankylosing spondylitis (Barrett et al (2008) Nat Genet. 40(8):955-62).

PsA is a frequent and chronic disease that encompasses a spectrum of overlapping clinical entities, including psoriasis and joint pain (Moll and Wright (1973) Semin Arthritis Rheum 3:55-78). About 10-40% of patients with psoriasis suffer from PsA. Recent efforts have been aimed at defining more stringent classification criteria for standardized recruitment into clinical trials (Taylor et al (2006) Arthritis Rheum 54:2665-73). PsA is associated with significant morbidity and disability, and thus constitutes a major socioeconomic burden. It is not only more common, but also more severe than previously thought (Gladman D D (2004) Psoriatic arthritis. In: Harris et al, eds. Kelly's Textbook of Rheumatology. 7th ed. Philadelphia: Saunders, p. 1154-64). The majority of patients will have psoriasis before the associated arthritis occurs and will be under treatment for their skin disease. NSAIDs are used for musculoskeletal pain symptoms.

Traditional disease modifying anti-rheumatic drugs (DMARDs) include methotrexate (MTX), sulfasalazine, cyclopsorine, and leflunomide and are inadequate for a number of patients because these drugs only partially control established disease (Mease P J (2008) Psoriatic Arthritis. In: Klippel et al, eds. Primer on Rheumatic Diseases. 13th ed. New York: Springer Science, p. 170-192). Several lines of evidence support the notion of prominent T cell involvement in the pathogenesis of PSA. Memory CD4+ and CD8+ cells are present in skin lesions as well as the inflamed synovium that express activation markers and have characteristics of oligoclonal expansion. (Curran et al (2004) J Immunol 172:1935-44 1935-44; Tassiulas et al (1999) Hum Immunol 60:479-491). Clinical trials have demonstrated the efficacy of T cell targeted therapy in PsA (cyclosporine A, CTLA4 Ig, alefacept). TNF blocking therapy was also successfully introduced to the treatment of patients with PsA (Mease P J et al. (2000) Lancet 356:385-90). Despite these efforts, an unmet clinical need exists for patients with PsA for better disease control and long term prevention of structural damage beyond mere abrogation of inflammatory processes. In addition, current treatment options for patients with intolerance or an inadequate response to anti-TNF-α agents are limited.

Secukinumab (AIN457) is a high-affinity fully human monoclonal anti-human antibody that inhibits Interleukin-17A activity. In a recent PsA proof-of-concept (PoC) study (AIN457A2206) (Example 1), secukinumab has emerged as a potential treatment for patients with PSA. However, since patient response to biological treatment is variable and it is desirable to avoid providing drug to patients who will be resistant thereto, there is a need to develop methods of treating PsA that first identity those patients most likely to benefit from a chosen biological treatment.

### BRIEF SUMMARY OF THE DISCLOSURE

While several single nucleotide polymorphisms (SNPs) are linked to the PsA disease state (Strange et al (2010) Nat. Genet. 42(11) 985-990; Huffmeier et al. (2010) Nat. Genet 42(11) 996-9; Ellinghaus et al. (2010) Nat. Genet 42(11) 991-5), thus far no biomarker has been identified as being predictive of whether a PsA patient will respond to a particular drug, e.g., an IL-17 antagonist. Provided herein are novel predictive methods and personalized therapies for treating PsA that maximize the benefit and minimize the risk of IL-17 antagonism in the PsA population by identifying those patients most likely to respond favorably to antagonism of IL-17 during treatment of PsA. This finding is based, in part, on the determinations that:

1) PsA patients carrying at least one rs240993 “T” allele (referred to herein as the “PsA non-response allele”), which is linked to TRAF3IP2 (TRAF3 interacting protein 2), display reduced response relative to PsA patients that do not carry any rs240993 “T” allele (i.e., patients homozygous for the rs240993 “C” allele);

2) PsA patients carrying at least one HLA-DRB1*04 allele (referred to herein as a “PsA response allele”) display improved response to secukinumab relative to PsA patients that do not carry any HLA-DRB1*04 allele; and

3) PsA patients carrying at least one TNFSF15 (Tumor necrosis factor (ligand) superfamily, member 15) rs4263839 “A” allele (also referred to herein as a “PsA response allele”) display improved response to secukinumab relative to PsA patients that do not carry any rs4263839 “A” allele.

We thus contemplate that testing subjects for the presence of at least one PsA non-response allele and/or at least one PsA response allele will be useful in a variety of pharmacogenetic products and methods that involve identifying individuals more likely to respond to IL-17 antagonsim and in helping physicians decide whether to prescribe IL-17 antagonists (e.g., secukinumab) to a patient having PsA. Accordingly, it is one object of the disclosure to provide methods of treating PsA, by administering the patient a therapeutically effective amount of an IL-17 antagonist, e.g., an IL-17 antibody, such as secukinumab, provided that the patient does not have a PsA non-response allele or provided that the patient has a PsA response allele. It is another object of the disclosure to provide methods of identifying patients who are more likely to respond to treatment of PsA with an IL-17 antagonist, e.g., an IL-17 antibody, such as the AINI457 antibody (secukinumab) by determining whether the patient has a PsA non-response allele or a PsA response allele. It is another object of the disclosure to provide methods of determining the likelihood that a PsA patient will respond to treatment with an IL-17 antagonist, e.g., an IL-17 antibody, such as secukinumab, by determining whether the patient has the presence of a PsA non-response allele or a PsA response allele.

Based upon the above objects and discoveries, disclosed herein are various methods of selectively treating a patient having PsA. In some embodiments, these methods comprise assaying a biological sample from the patient for the presence (or absence) of a PsA non-response allele or a PsA response allele; and thereafter selectively administering a therapeutically effective amount of an IL-17 antagonist, e.g., secukinumab, to the patient if the patient does not have the PsA non-response allele or if the patient has a PsA response allele.

Disclosed herein are also various methods of predicting the likelihood that a patient having PsA will respond to treatment with an IL-17 antagonist, e.g., secukinumab. In some embodiments, these methods comprise assaying a biological sample from the patient for the presence of a PsA non-response allele, wherein the presence of the PsA non-response allele is indicative of a decreased likelihood that the patient will respond to treatment with the IL-17 antagonist. In some embodiments, these methods comprise assaying a biological sample from the patient for the presence of a PsA response allele, wherein the presence of the PsA response allele is indicative of an increased likelihood that the patient will respond to treatment with the IL-17 antagonist.

In preferred embodiments, the IL-17 antagonist is an IL-17 binding molecule, preferably a human antibody, most preferably secukinumab.

Additional methods, uses, and kits are provided in the the following description and appended claims. Further features, advantages and aspects of the present disclosure will become apparent to those skilled in the art from the following description and appended claims.

### BRIEF DESCRIPTION OF THE DRAWINGS

FIG. 1 shows the CAIN457A2206 clinical trial design.

FIG. 2 shows a region across REV3L and TRAF3IP2 having high linkage disequilibrium (LD), which suggests that SNP rs240993 may actually be ‘tagging’ a causal SNP in TRAF3IP2.

### DETAILED DESCRIPTION OF THE DISCLOSURE

The term “comprising” encompasses “including” as well as “consisting,” e.g. a composition “comprising” X may consist exclusively of X or may include something additional, e.g., X+Y.

The term “assaying” is used to refer to the act of identifying, screening, probing, testing measuring or determining, which act may be performed by any conventional means. For example, a sample may be assayed for the presence of a particular genetic or protein marker by using an ELISA assay, a Northern blot, imaging, serotyping, cellular typing, gene sequencing, phenotyping, haplotyping, immunohistochemistry, western blot, mass spectrometry, etc. The term “detecting” (and the like) means the act of extracting particular information from a given source. The terms “assaying” and “determining” contemplate a transformation of matter, e.g., a transformation of a biological sample, e.g., a blood sample or other tissue sample, from one state to another by means of subjecting that sample to physical testing.

The term “obtaining” means to procure, e.g., to acquire possession of in any way, e.g., by physical intervention (e.g., biopsy, blood draw) or non-physical intervention (e.g, transmittal of information via a server), etc.

The phrase “assaying a biological sample . . . ” and the like is used to mean that a sample may be tested (either directly or indirectly) for either the presence or absence of a given PsA non-response allele or PsA response allele). It will be understood that, in a situation where the presence of a substance denotes one probability and the absence of a substance denotes a different probability, then either the presence or the absence of such substance may be used to guide a therapeutic decision. For example, one may determine if a patient has a PsA non-response allele by determining the actual existence of a PsA non-response allele in the genome of a patient or by determining the absence of the PsA non-response allele in the genome of a patient. In both such cases, one has determined whether the patient has the presence of the PsA non-response allele. The disclosed methods involve, inter alia, determining whether a particular individual has a PsA non-response allele or a PsA response allele. This determination is undertaken by identifying whether the patient has the presence of an rs240993 non-response allele, an HLA-DRB1*04 allele, or an rs4263839 response allele. Each of these determinations (i.e., presence or absence), on its own, provides the allelic status of the patient and thus each of these deteriminations equally provide an indication of whether a particular individual would or would not respond more favorably to IL-17 antagonism.

It will be understood that patients heterozygous or homozygous for the PsA non-response alleles disclosed herein (rs240993 non-response allele) are less likely to respond favorably to IL-17 antagonism. Thus, to provide an indication of decreased responsiveness, a biological sample need only be assayed for one PsA non-response allele, but clearly may be assayed for both PsA non-response alleles. Similarly, it will be understood that patients heterozygous or homozygous for the PsA response alleles disclosed herein (HLA-DRB1*04 allele and rs4263839 response allele) are more likely to respond favorably to IL-17 antagonism. Thus, to provide an indication of increased responsiveness, a biologically sample need only be assayed for one PsA response allele, but clearly may be assayed for both PsA response alleles.

The term “about” in relation to a numerical value x means +/−10% unless the cotext dictates otherwise. The word “substantially” does not exclude “completely,” e.g., a composition which is “substantially free” from Y may be completely free from Y. Where necessary, the word “substantially” may be omitted from the definition of the disclosure.

“IL-17 antagonist” as used herein refers to a molecule capable of antagonizing (e.g., reducing, inhibiting, decreasing, delaying) IL-17 function, expression and/or signalling (e.g., by blocking the binding of IL-17 to the IL-17 receptor). Non-limiting examples of IL-17 antagonists include IL-17 binding molecules and IL-17 receptor binding molecules. In some embodiments of the disclosed methods, regimens, kits, processes, uses and compositions, an IL-17 antagonist is employed.

By “IL-17 binding molecule” is meant any molecule capable of binding to the human IL-17 antigen either alone or associated with other molecules. The binding reaction may be shown by standard methods (qualitative assays) including, for example, a binding assay, competition assay or a bioassay for determining the inhibition of IL-17 binding to its receptor or any kind of binding assays, with reference to a negative control test in which an antibody of unrelated specificity, but ideally of the same isotype, e.g., an anti-CD25 antibody, is used. Non-limiting examples of IL-17 binding molecules include small molecules, IL-17 receptor decoys, and antibodies that bind to IL-17 as produced by B-cells or hybridomas and chimeric, CDR-grafted or human antibodies or any fragment thereof, e.g., F(ab′)₂ and Fab fragments, as well as single chain or single domain antibodies. Preferably the IL-17 binding molecule antagonizes (e.g., reduces, inhibits, decreases, delays) IL-17 function, expression and/or signalling. In some embodiments of the disclosed methods, regimens, kits, processes, uses and compositions, an IL-17 binding molecule is employed.

By “IL-17 receptor binding molecule” is meant any molecule capable of binding to the human IL-17 receptor either alone or associated with other molecules. The binding reaction may be shown by standard methods (qualitative assays) including, for example, a binding assay, competition assay or a bioassay for determining the inhibition of IL-17 receptor binding to IL-17 or any kind of binding assays, with reference to a negative control test in which an antibody of unrelated specificity, but ideally of the same isotype, e.g., an anti-CD25 antibody, is used. Non-limiting examples of IL-17 receptor binding molecules include small molecules, IL-17 decoys, and antibodies to the IL-17 receptor as produced by B-cells or hybridomas and chimeric, CDR-grafted or human antibodies or any fragment thereof, e.g., F(ab′)₂ and Fab fragments, as well as single chain or single domain antibodies. Preferably the IL-17 receptor binding molecule antagonizes (e.g., reduces, inhibits, decreases, delays) IL-17 function, expression and/or signalling. In some embodiments of the disclosed methods, regimens, kits, processes, uses and compositions, an IL-17 receptor binding molecule is employed.

The term “antibody” as referred to herein includes whole antibodies and any antigen-binding portion or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The V_{H} and V_{L} regions can be further subdivided into regions of hypervariability, termed hypervariable regions or complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. In some embodiments of the disclosed methods, regimens, kits, processes, uses and compositions, an antibody to IL-17 or the IL-17 receptor is employed, preferably an antibody to IL-17, e.g., secukinumab.

The term “antigen-binding portion” of an antibody as used herein, refers to fragments of an antibody that retain the ability to specifically bind to an antigen (e.g., IL-17). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, CL and CH1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated CDR. Exemplary antigen binding sites include the CDRs of secukinumab as set forth in SEQ ID NOs:1-6 and 11-13 (Table 3), preferably the heavy chain CDR3. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies are also intended to be encompassed within the term “antibody”. Single chain antibodies and antigen-binding portions are obtained using conventional techniques known to those of skill in the art. In some embodiments of the disclosed methods, regimens, kits, processes, uses and compositions, a single chain antibody or an antigen-binding portion of an antibody against IL-17 (e.g., secukinumab) or the IL-17 receptor is employed.

An “isolated antibody”, as used herein, refers to an antibody that is substantially free of other antibodies having different antigenic specificities (e.g., an isolated antibody that specifically binds IL-17 is substantially free of antibodies that specifically bind antigens other than IL-17). The term “monoclonal antibody” or “monoclonal antibody composition” as used herein refer to a preparation of antibody molecules of single molecular composition. The term “human antibody”, as used herein, is intended to include antibodies having variable regions in which both the framework and CDR regions are derived from sequences of human origin. A “human antibody” need not be produced by a human, human tissue or human cell. The human antibodies of the disclosure may include amino acid residues not encoded by human sequences (e.g., mutations introduced by random or site-specific mutagenesis in vitro, by N-nucleotide addition at junctions in vivo during recombination of antibody genes, or by somatic mutation in vivo). In some embodiments of the disclosed methods, regimens, kits, processes, uses and compositions, the IL-17 antagonist is a human antibody, an isolated antibody, and/or a monoclonal antibody.

The term “IL-17” refers to IL-17A, formerly known as CTLA8, and includes wild-type IL-17A from various species (e.g., human, mouse, and monkey), polymorphic variants of IL-17A, and functional equivalents of IL-17A. Functional equivalents of IL-17A according to the present disclosure preferably have at least about 65%, 75%, 85%, 95%, 96%, 97%, 98%, or even 99% overall sequence identity with a wild-type IL-17A (e.g., human IL-17A), and substantially retain the ability to induce IL-6 production by human dermal fibroblasts.

The term “K_{D}” is intended to refer to the dissociation rate of a particular antibody-antigen interaction. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system. In some embodiments, the IL-17 antagonist, e.g., IL-17 binding molecule (e.g., IL-17 antibody or antigen binding fragment thereof, e.g., secukinumab) or IL-17 receptor binding molecule (e.g., IL-17 receptor antibody or antigen binding fragment thereof) binds human IL-17 with a K_{D} of about 100-250 pM.

The term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. Standard assays to evaluate the binding affinity of the antibodies toward IL-17 of various species are known in the art, including for example, ELISAs, western blots and RIAs. The binding kinetics (e.g., binding affinity) of the antibodies also can be assessed by standard assays known in the art, such as by Biacore analysis.

As used herein, the terms “subject” and “patient” include any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

An antibody that “inhibits” one or more of these IL-17 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (or when a control antibody of irrelevant specificity is present). An antibody that inhibits IL-17 activity affects a statistically significant decrease, e.g., by at least 10% of the measured parameter, by at least 50%, 80% or 90%, and in certain embodiments of the disclosed methods, uses, processes, kits and compositions, the IL-17 antibody used may inhibit greater than 95%, 98% or 99% of IL-17 functional activity.

“Inhibit IL-6” as used herein refers to the ability of an IL-17 antagonist (e.g., secukinumab) to decrease IL-6 production from primary human dermal fibroblasts. The production of IL-6 in primary human (dermal) fibroblasts is dependent on IL-17 (Hwang et al., (2004) Arthritis Res Ther; 6:R120-128). In short, human dermal fibroblasts are stimulated with recombinant IL-17 in the presence of various concentrations of an IL-17 binding molecule or human IL-17 receptor with Fc part. The chimeric anti-CD25 antibody Simulect® (basiliximab) may be conveniently used as a negative control. Supernatant is taken after 16 h stimulation and assayed for IL-6 by ELISA. An IL-17 antagonist, e.g., IL-17 binding molecule (e.g., IL-17 antibody or antigen binding fragment thereof, e.g., secukinumab) or IL-17 receptor binding molecule (e.g., IL-17 antibody or antigen binding fragment thereof) as disclosed herein typically has an IC₅₀ for inhibition of IL-6 production (in the presence 1 nM human IL-17) of about 50 nM or less (e.g., from about 0.01 to about 50 nM) when tested as above, i.e., said inhibitory activity being measured on IL-6 production induced by hu-IL-17 in human dermal fibroblasts. In some embodiments of the disclosed methods, regimens, kits, processes, uses and compositions, IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof) and functional derivatives thereof have an IC₅₀ for inhibition of IL-6 production as defined above of about 20 nM or less, more preferably of about 10 nM or less, more preferably of about 5 nM or less, more preferably of about 2 nM or less, more preferably of about 1 nM or less.

The term “derivative”, unless otherwise indicated, is used to define amino acid sequence variants, and covalent modifications (e.g., pegylation, deamidation, hydroxylation, phosphorylation, methylation, etc.) of an IL-17 antagonist, e.g., IL-17 binding molecule (e.g., IL-17 antibody or antigen binding fragment thereof, e.g., secukinumab) or IL-17 receptor binding molecule (e.g., IL-17 receptor antibody or antigen binding fragment thereof) according to the present disclosure, e.g., of a specified sequence (e.g., a variable domain). A “functional derivative” includes a molecule having a qualitative biological activity in common with the disclosed IL-17 antagonists, e.g., IL-17 binding molecules. A functional derivative includes fragments and peptide analogs of an IL-17 antagonist as disclosed herein. Fragments comprise regions within the sequence of a polypeptide according to the present disclosure, e.g., of a specified sequence. Functional derivatives of the IL-17 antagonists disclosed herein (e.g., functional derivatives of secukinumab) preferably comprise V_{H} and/or V_{L} domains that have at least about 65%, 75%, 85%, 95%, 96%, 97%, 98%, or even 99% overall sequence identity with the V_{H} and/or V_{L} sequences of the IL-17 binding molecules disclosed herein (e.g., the V_{H} and/or V_{L} sequences of Table 3), and substantially retain the ability to bind human IL-17 or, e.g., inhibit IL-6 production of IL-17 induced human dermal fibroblasts.

The phrase “substantially identical” means that the relevant amino acid or nucleotide sequence (e.g., V_{H} or V_{L} domain) will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region (e.g., V_{H} or V_{L} domain). In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity of a derivative IL-17 antibody (e.g., a derivative of secukinumab, e.g., a secukinumab biosimilar antibody) can be about 90% or greater, e.g., 90%, 91%, 92%, 93%, 94%, 95%, 96%, 97%, 98%, 99% or higher relative to the disclosed sequences.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are well known. The percent identity can be determined by standard alignment algorithms, for example, the Basic Local Alignment Search Tool (BLAST) described by Altshul et al. ((1990) J. Mol. Biol., 215: 403 410); the algorithm of Needleman et al. ((1970) J. Mol. Biol., 48: 444 453); or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM 120 weight residue table, a gap length penalty of 12 and a gap penalty of 4.

“Amino acid(s)” refer to all naturally occurring L-α-amino acids, e.g., and include D-amino acids. The phrase “amino acid sequence variant” refers to molecules with some differences in their amino acid sequences as compared to the sequences according to the present disclosure. Amino acid sequence variants of a polypeptide according to the present disclosure, e.g., of a specified sequence, still have the ability to bind the human IL-17 or, e g , inhibit IL-6 production of IL-17 induced human dermal fibroblasts. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The term “pharmaceutically acceptable” means a nontoxic material that does not interfere with the effectiveness of the biological activity of the active ingredient(s).

The term “administering” in relation to a compound, e.g., an IL-17 binding molecule or another agent, is used to refer to delivery of that compound to a patient by any route.

As used herein, a “therapeutically effective amount” refers to an amount of an IL-17 antagonist, e.g., IL-17 binding molecule (e.g., IL-17 antibody or antigen binding fragment thereof, e.g., secukinumab) or IL-17 receptor binding molecule (e.g., IL-17 antibody or antigen binding fragment thereof) that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., an IL-17 antagonist, e.g., secukinumab) administered alone, the term refers to that ingredient alone. When applied to a combination, the term refers to combined amounts of the active ingredients that result in the therapeutic effect, whether administered in combination, serially or simultaneously.

The term “treatment” or “treat” refer to both prophylactic or preventative treatment as well as curative or disease modifying treatment, including treatment of a patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment.

The phrase “respond to treatment” is used to mean that a patient, upon being delivered a particular treatment, e.g., an IL-17 binding molecule (e.g., secukinumab) shows a clinically meaningful benefit from said treatment. In the case of PsA, such benefit may be measured by a variety of criteria, e.g., ACR20, ACR50, ACR70, DAS28, etc. (see Example 1). All such criteria are acceptable measures of whether a PsA patient is responding to a given treatment. The phrase “respond to treatment” is meant to be construed comparatively, rather than as an absolute response. For example, a patient having a PsA non-response allele is predicted to have less benefit from treatment with an IL-17 antagonist than a patient who does not have a PsA non-response allele. Similarly, a patient having a PsA response allele is predicted to have more benefit from treatment with an IL-17 antagonist than a patient who does not have a PsA response allele. These non-carriers of PsA non-response alleles and carriers of PsA response alleles respond more favorably to treatment with the IL-17 antagonist, and may simply be said to “respond to treatment” with an IL-17 antagonist.

The phrase “receiving data” is used to mean obtaining possession of information by any available means, e.g., orally, electronically (e.g., by electronic mail, encoded on diskette or other media), written, etc.

As used herein, the phrase “psoriatic arthritis” and its abbreviation “PsA” refer to an immune-mediated chronic inflammatory disease belonging to the spectrum of conditions commonly referred to as seronegative spondylarthropathies (SpA). The CASPAR criteria (Taylor et al (2006) Arthritis Rheum 54:2665-73) may be used to diagnose a patient as having PsA.

As used herein, “selecting” and “selected” in reference to a patient is used to mean that a particular patient is specifically chosen from a larger group of patients on the basis of (due to) the particular patient having a predetermined criteria, e.g., the patient does not have a PsA non-response allele or the patient has a PsA response allele. Similarly, “selectively treating a patient having PsA” refers to providing treatment to a PsA patient that is specifically chosen from a larger group of patients on the basis of (due to) the particular patient having a predetermined criteria, e.g., the patient does not have a PsA non-response allele or the patient has a PsA response allele. Similarly, “selectively administering” refers to administering a drug to a PsA patient that is specifically chosen from a larger group of patients on the basis of (due to) the particular patient having a predetermined criteria, e.g., a particular genetic or other biological marker. By selecting, selectively treating and selectively administering, it is meant that a patient is delivered a personalized therapy for PsA based on the patient's biology, rather than being delivered a standard treatment regimen based solely on having the PsA disease.

As used herein, “predicting” indicates that the methods described herein provide information to enable a health care provider to determine the likelihood that an individual having PsA will respond to or will respond more favorably to treatment with an IL-17 binding molecule. It does not refer to the ability to predict response with 100% accuracy. Instead, the skilled artisan will understand that it refers to an increased probability.

As used herein, “likelihood” and “likely” is a measurement of how probable an event is to occur. It may be used interchangably with “probability”. Likelihood refers to a probability that is more than speculation, but less than certainty. Thus, an event is likely if a reasonable person using common sense, training or experience concludes that, given the circumstances, an event is probable. In some embodiments, once likelihood has been ascertained, the patient may be treated (or treatment continued, or treatment proceed with a dosage increase) with the IL-17 binding molecule or the patient may not be treated (or treatment discontinued, or treatment proceed with a lowered dose) with the IL-17 binding molecule.

The phrase “increased likelihood” refers to an increase in the probability that an event will occur. For example, some methods herein allow prediction of whether a patient will display an increased likelihood of responding to treatment with an IL-17 binding molecule or an increased likelihood of responding better to treatment with an IL-17 binding molecule in comparison to a a PsA patient who has a PsA non-response allele or a PsA patient who does not have a PsA response allele.

The phrase “decreased likelihood” refers to a decrease in the probability that an event will occur. For example, the methods herein allow prediction of whether a patient will display a decreased likelihood of responding to treatment with an IL-17 binding molecule or a decreased likelihood of responding better to treatment with an IL-17 binding molecule in comparison to a PsA patient who does not have a PsA non-response allele or a PsA patient who does not have a PsA response allele.

As used herein “SNP” refers to “single nucleotide polymorphism”. A single nucleotide polymorphism is a DNA sequence variation occurring when a single nucleotide in the genome (or other shared sequence) differs between members of a biological species or paired chromosomes in an individual. Most SNPs have only two alleles, and one is usually more common in the population. A SNP may be present in an exon or an intron of a gene, an upstream or downstream untranslated region of a gene, or in a purely genomic location (i.e., non-transcribed). When a SNP occurs in the coding region of a gene, the SNP may be silent (i.e., a synonymous polymorphism) due to the redundancy of the genetic code, or the SNP may result in a change in the sequence of the encoded polypeptide (i.e., a non-synonymous polymorphism). In the instant disclosure, SNPs are identified by their Single Nucleotide Polymorphism Database (dbSNP) rs number, e.g., rs4263839. The dbSNP is a free public archive for genetic variation within and across different species developed and hosted by the National Center for Biotechnology Information (NCBI) in collaboration with the National Human Genome Research Institute (NHGRI).

A polymorphic site, such as a SNP, is usually preceded by and followed by conserved sequences in the genome of the population of interest and thus the location of a polymorphic site can often be made in reference to a consensus nucleic acid sequence (e.g., of thirty to sixty nucleotides) that bracket the polymorphic site, which in the case of a SNP is commonly referred to as the “SNP context sequence”. Context sequences for the SNPs disclosed herein may be found in the NCBI SNP database available at: www.ncbi.nlm.nih.gov/snp. Alternatively, the location of the polymorphic site may be identified by its location in a reference sequence (e.g., GeneBank deposit) relative to the start of the gene, mRNA transcript, BAC clone or even relative to the initiation codon (ATG) for protein translation. The skilled artisan understands that the location of a particular polymorphic site may not occur at precisely the same position in a reference or context sequence in each individual in a population of interest due to the presence of one or more insertions or deletions in that individual as compared to the consensus or reference sequence. It is routine for the skilled artisan to design robust, specific and accurate assays for detecting the alternative alleles at a polymorphic site in any given individual, when the skilled artisan is provided with the identity of the alternative alleles at the polymorphic site to be detected and one or both of a reference sequence or context sequence in which the polymorphic site occurs. Thus, the skilled artisan will understand that specifying the location of any polymorphic site described herein by reference to a particular position in a reference or context sequence (or with respect to an initiation codon in such a sequence) is merely for convenience and that any specifically enumerated nucleotide position literally includes whatever nucleotide position the same polymorphic site is actually located at in the same locus in any individual being tested for the presence or absence of a genetic marker of the invention using any of the genotyping methods described herein or other genotyping methods well-known in the art.

As shown in the Examples, we have determined that PsA patients carrying at least one rs240993 “T” allele (referred to herein as the “PsA non-response allele”), which is linked to TRAF3IP2 (TRAF3 interacting protein 2), display reduced response relative to PsA patients that do not carry any rs240993 “T” allele. The rs240993 polymorphism is in the REV3L gene, which is downstream of TRAF3IP2. As used herein, “REV3L” refers to the human REV3L gene (also known as “REV3”), which encodes a ˜350-kDa protein (REV3), the catalytic subunit of DNA polymerase ζ. As used herein, “TRAF3IP2” refers to the human TRAF3IP2 gene, which encodes ACT1 (also known as Adapter protein CIKS), a protein that interacts with TRAF proteins (tumor necrosis factor receptor-associated factors), e.g., TRAF3 and TRAF6, to activate either NF-kappaB or Jun kinase. (see, e.g., Wu et al. (2010) Adv. Exp. Med. Biol. 946:223-35). ACT1 also play an important role in IL-17 signaling. For example, Qian et al. (2007) Nat. Immunol. 8(3)247-56 found that ACT1 is recruited to the IL-17R following stimulation with IL-17, and that abolishing ACT1 in astroglial and gut epithelial cells lead to a reduction in IL-17 induced expression of inflammation-related genes. In a recent report by Zhu et al. (2010) J. Exp. Med. 207(12)2647-2662, it was reported that TRAF3 is a receptor proximal negative regulator of IL-17 receptor (IL-17R) signaling. Zhu et al. showed that TRAF3 greatly suppressed IL-17-induced NF-kappaB and mitogen-activated protein kinase activation and subsequent production of inflammatory cytokines and chemokines

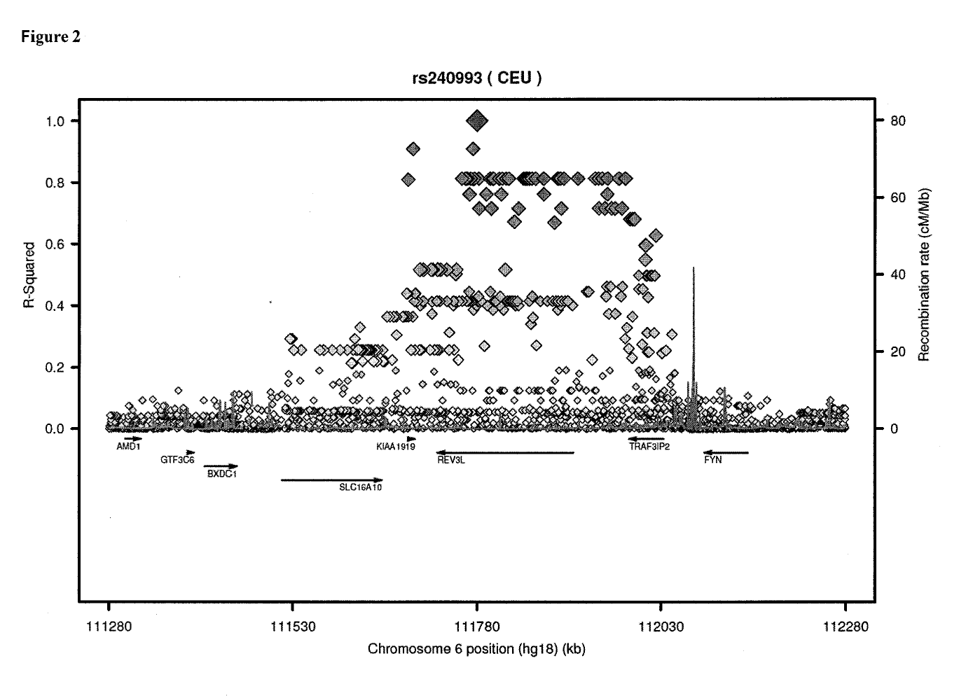

As shown in FIG. 2, there is a region across REV3L and TRAF3IP2 showing high linkage disequilibrium (LD), suggesting that rs240993 may be ‘tagging’ a causal SNP in TRAF3IP2. In a recent publication, Strange et al (2010) Nat. Genet. 42(11) 985-990 report that rs240993 T allele, which is in the chromosome 6q21 region, is associated with psoriasis disease risk (see also Chandran (2012) Clinic Rev Allerg Immunol. DOI: 10.1007/s12016-012-8303-5). The authors note that within this chromosomal region are four known genes, with TRAF3IP2 being noteworthy for its involvement in the IL-17 dependent NF-kappabeta activation and Th17-mediated inflammatory responses. Certain other TRAF3IP2 SNPs have also been shown to be associated with PsA and psoriasis (Huffmeier et al. (2010) Nat. Genet 42(11) 996-9; Ellinghaus et al. (2010) Nat. Genet 42(11) 991-5).

As used herein, “rs240993” refers to a T/C/A/G SNP located within an intron of the human REV3L gene (GeneBank Accession No. NM_{—}002912.3). The rs240993 polymorphic site is located at chromosomal position 111673714 (NCBI genome build 37.3; GRCh37.p5), which is position 15843171 of Contig NT_{—}025741.15.

The phrase “PsA non-response allele” as used herein refers to the T allele (A allele, in the case of the noncoding strand) at the rs240993 polymorphic site (also referred to as the “rs240993 non-response allele”). In some embodiments of the disclosed methods, uses, and kits, the patient has at least one PsA non-response allele.

As shown in the Examples, we have determined that PsA patients carrying at least one TNFSF15 (Tumor necrosis factor (ligand) superfamily, member 15) rs4263839 “A” allele (also referred to herein as a “PsA response allele”) display improved response to secukinumab relative to PsA patients that do not carry any rs4263839 “A” allele. “TNFSF15” refers to the human Tumor necrosis factor (ligand) superfamily member 15 gene, which encodes TNF superfamily ligand TL1A (also known as VEGI). TL1A is a cytokine that belongs to the tumor necrosis factor (TNF) ligand family and is abundantly expressed in endothelial cells. (Sethi et al. (2009) Adv. Exp. Med. Biol. 647:207-15). TL1A expression is inducible by inflammatory stimuli, e.g., TNF and IL-1 alpha, and in turn activates multiple cell signaling pathways including NF-kappaB, STAT3, JNK, p38 MAPK and p42/p44 MAPK. (Sethi et al.) VEGI suppresses the proliferation of endothelial cells and tumor cells, induces maturation of dendritic cells and induces osteoclastogenesis. (Sethi et al). Binding of TL1A to death receptor 3 (DR3) on activated CD4 T cells provides a co-stimulatory signal that amplifies the inflammatory response by inducing proliferation and differentiation of T-helper 17 cells, with production of interferon-gamma and IL-17. (Pappu et al. (2008) J Exp Med 205:1049-62; Meylan et al. (2008) Immunity 29:79-89). The TNFSF15 gene is found on chromosome 9.

As used herein, “rs4263839” refers to an A/G SNP located within an intron of the human TNFSF15 gene in a region thought to be associated with transcriptional regulation in several cell lines (Zucchelli et al. (2011) Gut 60(12):1671-1). The G allele of rs4263839 is associated with an increased risk (odds ratio 1.37) of inflammatory bowel syndrome. (Zucchelli et al.; Barrett et al. (2008) Nat Genet. 40(8):955-62). The rs4263839 polymorphic site is located at position 117566440 of GRCh37.p5, which is position 46730972 of Contig NT_{—}008470.19, which is position 6969 of the human TNFSF15 gene set forth as GeneBank Accession No. NG_{—}011488.2.

As shown in the Examples, we have determined that PsA patients carrying at least one HLA-DRB1*04 allele (referred to herein as a “PsA response allele”) display improved response to secukinumab relative to PsA patients that do not carry any HLA-DRB1*04 allele. “HLA” refers to human leukocyte antigen. The HLA is located on chromosome 6p21.31 and covers a region of about 3.6 Mbp depending on the haplotype. HLA molecules are coded by three groups of genes, HLA class I, HLA class II and HLA class III genes. HLA class I proteins are coded by the genes HLA-A, HLA-B, and HLA-C. HLA class II proteins are coded by the genes HLA-DR, HLA-DQ, HLA-DP, HLA-DM, HLA-DOA and HLA-DOB. The HLA class II proteins are part of the complement system. The polymorphic HLA class I genes HLA-A, -B, and -C and class II genes HLA-DR, -DQ and -DP encode various proteins (see, e.g., hla.alleles.org/proteins/class2.html) and various antigens (see, e.g., hla.alleles.org/antigens/recognised_serology.html).

HLA class II molecules consist of two transmembrane polypeptides, the alpha and beta chain. The beta chain is more polymorphic compared to the alpha chain, and HLA typing is generally undertaken on beta chains (e.g., HLA-DRB1 to DRB9). HLA allele naming is made according to the 2010 WHO Nomenclature Committee for Factors of the HLA System (Marsh et al. (2010) Tissue Antigens 75:291-455; Marsh et al. (2010) Bone Marrow Transplantation 45:846-8). Several digits are used to identify the HLA allele. The particular HLA locus (HLA-A, HLA-B, HLA-C, HLA-DR, HLA-DQ and HLA-DP) is separated by the symbol * from two numeric digits, which assigns the serologic equivalent of the antigen (this level describes the “type” or “allelic group”). As an example, HLA-DRB1*04 represents an allelic group from the HLA-DRB1 locus. This “two digit” resolution denotes a group of alleles (e.g., a group of alleles from the HLA-DRB1 locus) consisting of various alleles that either encode a similar antigen (e.g, the HLA-DR4 serologic antigen) or that share high sequence homology. This is followed by a colon and another two or three numeric digits, which identifies the specific encoded protein (this level describes the “subtype” or “allelic subtype”). As an example, HLA-DRB1*04:01 is a specific allele within the HLA-DRB1*04 allelic group that encodes a HLA-DR beta chain having a specific amino acid sequence. This “four digit” resolution denotes a particular genomic sequence variation within an allelic group that results in differences in the amino acid sequence of the encoded polypeptide product. Alleles can be further defined using additional colons and numerals that indicate synonymous DNA substitution within the coding region of the allele or that indicate DNA differences in a non-coding region (9 digit level).

“HLA-DRB1*04 allelic group” refers to the allelic group (or type) from the HLA-DRB1 locus consisting of various alleles that either encode the HLA-DR4 serologic antigen or that share high sequence homology.

“HLA-DRB1*04 allele” or “allele in the HLA-DRB1*04 allelic group” refers to an allele within the HLA-DRB1*04 allelic group, e.g., HLA-DRB1*04:01, HLA-DRB1*04:05, etc. Nonlimiting IMG/HLA Database (part of the EMBL-EBI database) reference numbers for exemplary HLA-DRB1*04 alleles are shown in Table 1, the sequences of which are accessible via www.ebi.ac.uk/imgt/hla/nomenclature/index.html.

TABLE 1
IMG/HLA Database reference numbers for HLA-DRB1*04
alleles. This list is not exhaustive.

Accession # Allele
HLA00685 DRB1*04:01:01
HLA00686 DRB1*04:01:02
HLA03066 DRB1*04:01:03
HLA04661 DRB1*04:01:04
HLA04663 DRB1*04:01:05
HLA04664 DRB1*04:01:06
HLA00687 DRB1*04:02
HLA00688 DRB1*04:03:01
HLA01009 DRB1*04:03:02
HLA02717 DRB1*04:03:03
HLA03172 DRB1*04:03:04
HLA04660 DRB1*04:03:05
HLA00689 DRB1*04:04:01
HLA04039 DRB1*04:04:02
HLA04659 DRB1*04:04:03
HLA04662 DRB1*04:04:04
HLA04710 DRB1*04:04:05
HLA00690 DRB1*04:05:01
HLA00691 DRB1*04:05:02
HLA01551 DRB1*04:05:03
HLA01605 DRB1*04:05:04
HLA03055 DRB1*04:05:05
HLA03375 DRB1*04:05:06
HLA04012 DRB1*04:05:07
HLA04035 DRB1*04:05:08
HLA04654 DRB1*04:05:09
HLA04857 DRB1*04:05:10
HLA00692 DRB1*04:06:01
HLA02172 DRB1*04:06:02
HLA04038 DRB1*04:06:03
HLA05777 DRB1*04:06:04
HLA00693 DRB1*04:07:01
HLA01453 DRB1*04:07:02
HLA01706 DRB1*04:07:03
HLA04658 DRB1*04:07:04
HLA00694 DRB1*04:08:01
HLA04008 DRB1*04:08:02
HLA00695 DRB1*04:09
HLA00696 DRB1*04:10
HLA00697 DRB1*04:11
HLA00698 DRB1*04:12
HLA00699 DRB1*04:13
HLA00700 DRB1*04:14
HLA00701 DRB1*04:15
HLA00702 DRB1*04:16
HLA00703 DRB1*04:17:01
HLA04408 DRB1*04:17:02
HLA00704 DRB1*04:18
HLA00705 DRB1*04:19
HLA00706 DRB1*04:20
HLA00707 DRB1*04:21
HLA00708 DRB1*04:22
HLA00709 DRB1*04:23
HLA00710 DRB1*04:24
HLA00711 DRB1*04:25
HLA00712 DRB1*04:26
HLA00713 DRB1*04:27
HLA00714 DRB1*04:28
HLA00715 DRB1*04:29
HLA00716 DRB1*04:30
HLA00717 DRB1*04:31
HLA00718 DRB1*04:32
HLA01088 DRB1*04:33
HLA01167 DRB1*04:34
HLA01235 DRB1*04:35
HLA01242 DRB1*04:36
HLA01338 DRB1*04:37
HLA01345 DRB1*04:38
HLA01458 DRB1*04:39
HLA01454 DRB1*04:40
HLA01459 DRB1*04:41
HLA01457 DRB1*04:42
HLA01499 DRB1*04:43
HLA01601 DRB1*04:44
HLA01695 DRB1*04:45
HLA01746 DRB1*04:46
HLA01780 DRB1*04:47
HLA01793 DRB1*04:48
HLA01810 DRB1*04:49
HLA01817 DRB1*04:50
HLA02039 DRB1*04:51
HLA02054 DRB1*04:52
HLA02146 DRB1*04:53
HLA02305 DRB1*04:54
HLA02306 DRB1*04:55
HLA02314 DRB1*04:56
HLA02460 DRB1*04:57
HLA02534 DRB1*04:58
HLA02580 DRB1*04:59
HLA02604 DRB1*04:60
HLA02705 DRB1*04:61
HLA02726 DRB1*04:62
HLA02741 DRB1*04:63
HLA02804 DRB1*04:64
HLA03028 DRB1*04:65
HLA03056 DRB1*04:66
HLA03060 DRB1*04:67
HLA03070 DRB1*04:68
HLA03071 DRB1*04:69
HLA03073 DRB1*04:70
HLA03074 DRB1*04:71
HLA03158 DRB1*04:72:01
HLA04673 DRB1*04:72:02
HLA03167 DRB1*04:73
HLA03296 DRB1*04:74
HLA03371 DRB1*04:75
HLA03372 DRB1*04:76
HLA03374 DRB1*04:77
HLA03585 DRB1*04:78
HLA03993 DRB1*04:79
HLA03998 DRB1*04:80
HLA04005 DRB1*04:81N
HLA04010 DRB1*04:82
HLA04036 DRB1*04:83
HLA04040 DRB1*04:84
HLA04349 DRB1*04:85
HLA04382 DRB1*04:86
HLA04383 DRB1*04:87
HLA04384 DRB1*04:88
HLA04672 DRB1*04:89
HLA05128 DRB1*04:90
HLA05146 DRB1*04:91
HLA05868 DRB1*04:92

These sequences are incorporated by reference herein in their entirety. “Product of an HLA-DRB1*04 allele” includes nucleic acid products of an HLA-DRB1*04 allele and polypeptide products of an HLA-DRB1*04 allele. “Polypeptide product of an HLA-DRB1*04 allele” refers to a polypeptide encoded by an HLA-DRB1*04 allele, a fragment of a polypeptide encoded by an HLA-DRB1*04 allele and the HLA-DR4 serologic antigen. “Nucleic acid product of an HLA-DRB1*04 allele” refers to any DNA (genomic, cDNA, etc.) or RNA products (e.g., pre-mRNA, mRNA, micro RNAs, etc.) of the HLA-DRB1*04 allele and fragments thereof.

“HLA-DR4 serotype” refers to the serotype of a patient expressing a polypeptide product of an HLA-DRB1*04 allele (e.g., a HLA-DR4 serologic antigen).

As used herein, both the A allele (T allele, in the case of the noncoding strand) at the rs4263839 polymorphic site (also called the “rs4263839 response allele”) and the HLA-DRB1*04 allelic group are collectively “PsA response alleles”. In some embodiments of the disclosed methods, uses, and kits, the patient has at least one PsA response allele, e.g., at least one rs4263839 response allele and/or at least one allele in the HLA-DRB1*04 allelic group.

As recognized by the skilled artisan, nucleic acid samples containing a particular SNP may be complementary double stranded molecules and thus reference to a particular site on the sense strand refers as well to the corresponding site on the complementary antisense strand. Similarly, reference to a particular genotype obtained for a SNP on both copies of one strand of a chromosome is equivalent to the complementary genotype obtained for the same SNP on both copies of the other strand. Thus, for example, a G/A genotype for the rs4263839 polymorphic site on the coding strand for the TNFSF15 gene is equivalent to a C/T genotype for that polymorphic site on the noncoding strand.

As used herein, the phrase “candidate PsA response marker” refers to the polymorphic sites and alleles shown in Table 2, which may be used to further stratify patients having an increased (or decreased) likelihood of responding to treatment with an IL-17 antagonist. It will be understood that the candidate PsA response markers in Table 2 can be used alone or in combination with the disclosed PsA non-response alleles and PsA response alleles to predict response of a PsA patient to an IL-17 binding molecule, e.g., secukinumab. In some embodiments, a biological sample from a patient is assayed for the presence of a PsA non-response allele and/or a PsA response allele and, optionally, a candidate PsA response marker.

TABLE 2
shows the gene, allele and position information
of the candidate PsA response markers.

| | | Alleles | Polymorphism |
|---|---|---|---|
| Variants | Gene of interest | (major/minor) | position |
| HLA-C*0602 | HLA-C | — | — |
| rs20541 | IL13 | A/C/G/T | non- |
| | | | synonymous |
| rs1974226 | IL17A | G/A | 3′ UTR |
| rs11209026 | IL23R | G/A | non- |
| | | | synonymous |
| rs2082412 | IL12B | A/G | downstream |
| rs17728338 | TNIP1 | A/G | upstream |
| rs610604 | TNFAIP3 | A/C | intronic |
| rs2066808 | STAT2/IL23A | C/T | intronic |
| rs2201841 | IL23R | T/C | intronic |
| rs495337 | SPATA2/ZNF313 | C/T | synonymous |
| rs4085613 | LCE3A/LCE3D | A/C/G/T | downstream |
| rs10484554 | HLA-C/HLA-B | C/T | upstream |
| rs7747909 | IL17A | A/G | 3′ UTR |
| rs30187 | ERAP1 | C/T | non- |
| | | | synonymous |
| rs27434 | ERAP1 | G/A | synonymous |
| rs27524 | ERAP1 | A/G | intronic |
| rs33980500 | TRAF3IP2 | C/T | non- |
| | | | synonymous |
| rs12188300 | IL12B | rs12188300 | upstream |

As used herein, “genomic sequence” refers to a DNA sequence present in a genome, and includes a region within an allele, an allele itself, or a larger DNA sequence of a chromosome containing an allele of interest.

Products of the PsA non-response alleles, candidate PsA response markers, and PsA response alleles include nucleic acid products and polypeptide products. “Polypeptide product” refers to a polypeptide encoded by a PsA non-response allele or PsA response allele and fragments thereof “Nucleic acid product” refers to any DNA (e.g., genomic, cDNA, etc.) or RNA (e.g., pre-mRNA, mRNA, miRNA, etc.) products of a PsA non-response allele or PsA response allele and fragments thereof.

An “equivalent genetic marker” refers to a genetic marker that is correlated to an allele of interest, e.g., it displays linkage disequilibrium (LD) or is in genetic linkage with the allele of interest. Equivalent genetic markers may be used to determine if a patient has a PsA non-response allele and/or a PsA response allele, rather than directly interrogating a biological sample from the patient for the PsA non-response allele and/or a PsA response allele per se. Various programs exist to help determine LD for particular SNPs, e.g, HaploBlock (available at bioinfo.cs.technion.ac.il/haploblock/), HapMap, WGA Viewer. Information on LD associated with rs4263839 may be found in Zucchelli et al. (2011) Gut 60(12):1671-1. An allele in the HLA-DRB1*04 allelic group can also be determined by detecting an equivalent genetic marker of an HLA-DRB*04 allele, which can be, e.g., a SNP (single nucleotide polymorphism), a microsatellite marker, another HLA allele (e.g., an HLA-DQB1 allele) or other kinds of genetic polymorphisms. For example, the presence of a genetic marker on the same haplotype as an HLA-DRB1*04 allele, rather than an HLA-DRB1*04 allele per se, may be indicative of a patient's likelihood for responding to treatment with an IL-17 binding molecule. A discussion of recombination and linkage disequilibrium within the HLA class II region is provided in Begovich et al. (1992) J. Immunology 148:249-58.

The term “probe” refers to any composition of matter that is useful for specifically detecting another substance, e.g., a substance related to a PsA non-response allele or a PsA response allele. A probe can be an oligonucleotide (including a conjugated oligonucleotide) that specifically hybridizes to a genomic sequence of a PsA non-response allele or a PsA response allele, or a nucleic acid product of a PsA non-response allele or a PsA response allele (e.g., genomic DNA or mRNA). A conjugated oligonucleotide refers to an oligonucleotide covalently bound to chromophore or molecules containing a ligand (e.g., an antigen), which is highly specific to a receptor molecule (e.g., an antibody specific to the antigen). The probe can also be a PCR primer, e.g., together with another primer, for amplifying a particular region within a PsA non-response allele or a PsA response allele. Further, the probe can be an antibody that specifically binds to polypeptide products of these alleles. Further, the probe can be any composition of matter capable of detecting (e.g., binding or hybridizing) an equivalent genetic marker of a PsA non-response allele or a PsA response allele. In preferred embodiments, the probe specifically hybridizes to a nucleic acid sequence (preferably genomic DNA) or specifically binds to a polypeptide sequence of an allele of interest.

The phrase “specifically hybridizes” is used to refer to hybrization under stringent hybridization conditions. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. One example of stringent hybridization conditions is hybridization in 6× sodium chloride/sodium citrate (SSC) at about 45° C., followed by at least one wash in 0.2×SSC, 0.1% SDS at 50° C. A second example of stringent hybridization conditions is hybridization in 6×SSC at about 45° C., followed by at least one wash in 0.2×SSC, 0.1% SDS at 55° C. Another example of stringent hybridization conditions is hybridization in 6×SSC at about 45° C., followed by at least one wash in 0.2×SSC, 0.1% SDS at 60° C. A further example of stringent hybridization conditions is hybridization in 6×SSC at about 45° C., followed by at least one wash in 0.2×SSC, 0.1% SDS at 65° C. High stringent conditions include hybridization in 0.5 M sodium phosphate, 7% SDS at 65° C., followed by at least one wash at 0.2×SSC, 1% SDS at 65° C.

The phrase “a region of a nucleic acid” is used to indicate a smaller sequence within a larger sequence of nucleic acids. For example, a gene is a region of a chromosome, an exon is a region of a gene, etc.

The term “specifically binds” in the context of polypeptides is used to mean that a probe binds a given polypeptide target (e.g., a polypeptide product of a PsA non-response allele) rather than randomly binding undesireable polypeptides. However, “specifically binds” does not exclude some cross reactivity with undesireable polypeptides, as long as that cross reactivity does not interfere with the capability of the probe to provide a a useful measure of the presence or absence of the given polypeptide target.

The term “capable” is used to mean that ability to achieve a given result, e.g., a probe that is capable of detecting the presence of a particular substance means that the probe may be used to detect the particular substance.

An “oligonucleotide” refers to a short sequence of nucleotides, e.g., 2-100 bases.

The term “biological sample” as used herein refers to a sample from a patient, which may be used for the purpose of identification, diagnosis, prediction, or monitoring. Preferred samples include synovial fluid, blood, blood-derived product (such as buffy coat, serum, and plasma), lymph, urine, tear, saliva, hair bulb cells, cerebrospinal fluid, buccal swabs, feces, synovial fluid, synovial cells, sputum, or tissue samples. In addition, one of skill in the art would realize that some samples would be more readily analyzed following a fractionation or purification procedure, for example, isolation of DNA from whole blood.

The phrases “has been previously treated for PsA” and “had a previous PsA treatment” and the like are used to mean a patient that has previously undergone PsA therapy, e.g, using an anti-PsA agent, e.g., the patient is a failure, an inadequate responder, or intolerant to a previous PsA therapy, anti-PsA agent or treatment regimen. Such patients include those previously treated with NSAIDs, DMARDs (e.g., methotrexate (MTX)), corticosteroids and/or biologics, such as TNF alpha antagonists, etc. The phrase “has not been previously treated for PsA” is used to mean a patient that has not previously undergone PsA treatment, i.e., the patient is “naïve.” As used herein, a patient that has not been previously treated for PsA with a TNF alpha antagonist is deemed “TNF alpha antagonist naive”. In some embodiments of the disclosed methods and uses, the patient has had a previous PsA treatment. In some embodiments of the disclosed methods and uses, the patient is TNF alpha antagonist naive.

As used herein, the phrase “PsA agent” refers to pharmaceuticals commonly prescribed for PsA patients, e.g., NSAIDs (e.g., indomethacin, naproxen, sulindac, diclofenac, aspirin, flurbiprofen, oxaprozin, salsalate, difunisal, piroxicam, etodolac, meclofenamate, ibuprophen, fenoprofen, ketoprofen, nabumetone, tolmetin, cholin magnesium salicylate, COX-2 inhibitors [e.g., celecoxib]), TNF alpha antagonists (etanercept, adalimumab, infliximab, golimumab), DMARDS (e.g., sulfasalazine, methotrexate), cyclosporin, retinoids and corticosteroids. In some embodiments of the disclosed methods and uses, the allelic status of a PsA patient drives a clinician to choose between two alternative therapies, i.e., treat the PsA patient with an IL-17 antagonist (e.g., secukinumab) or treat the patient with a different PsA agent (e.g., a DMARD).

The term “failure” to a previous PsA therapy refers to: (1) a patient who has no meaningful clinical benefit (primary lack of efficacy); (2) a patient who has a measurable and meaningful response, but for whom response could be better, e.g., low PsA disease activity or PsA remission was not achieved (also termed “inadequate response”); (3) a patient who, after an initial good response, worsens (secondary loss of efficacy); and (4) a patient who has a good response but discontinues because of a side effect (also termed “intolerance”). Patients who show a TNF alpha antagonist inadequate response (TNF-IR) or intolerance to a TNF alpha antagonist are considered TNF failures. Patients who show MTX inadequate response (MTX-IR) or intolerance to TMX are considered MTX failures. Patients who show DMARD inadequate response (DMARD-IR) or intolerance to DMARDs are considered DMARD failures. Patients who show NSAID inadequate response (NSAID-IR) or intolerance to NSAIDs are considered NSAID failures. In some embodiments of the disclosed methods and uses, the patient is a failure, an inadequate responder, or intolerant to a previous PsA treatment.

### IL-17 Antagonists

The various disclosed pharmaceutical compositions, regimens, processes, uses, methods and kits utilize an IL-17 antagonist, e.g., IL-17 binding molecule (e.g., IL-17 antibody or antigen binding fragment thereof, e.g., secukinumab) or IL-17 receptor binding molecule (e.g., IL-17 receptor antibody or antigen binding fragment thereof).

In one embodiment, the IL-17 antagonist, e.g., IL-17 binding molecule (e.g., IL-17 antibody or antigen binding fragment thereof, e.g., secukinumab) comprises at least one immunoglobulin heavy chain variable domain (V_{H}) comprising hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3. In one embodiment, the IL-17 antagonist, e.g., IL-17 binding molecule (e.g., IL-17 antibody or antigen binding fragment thereof, e.g., secukinumab) comprises at least one immunoglobulin light chain variable domain (V_{L}') comprising hypervariable regions CDR1′, CDR2′ and CDR3′, said CDR1′ having the amino acid sequence SEQ ID NO:4, said CDR2′ having the amino acid sequence SEQ ID NO:5 and said CDR3′ having the amino acid sequence SEQ ID NO:6. In one embodiment, the IL-17 antagonist, e.g., IL-17 binding molecule (e.g., IL-17 antibody or antigen binding fragment thereof, e.g., secukinumab) comprises at least one immunoglobulin heavy chain variable domain (V_{H}) comprising hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13.

In one embodiment, the IL-17 antagonist, e.g., IL-17 binding molecule (e.g., IL-17 antibody or antigen binding fragment thereof, e.g., secukinumab) comprises at least one immunoglobulin V_{H} domain and at least one immunoglobulin V_{L} domain, wherein: a) the immunoglobulin V_{H} domain comprises (e.g., in sequence): i) hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; or ii) hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13; and b) the immunoglobulin V_{L} domain comprises (e.g., in sequence) hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the IL-17 antagonist, e.g., IL-17 binding molecule (e.g., IL-17 antibody or antigen binding fragment thereof, e.g., secukinumab) comprises: a) an immunoglobulin heavy chain variable domain (V_{H}) comprising the amino acid sequence set forth as SEQ ID NO:8; b) an immunoglobulin light chain variable domain (V_{L}) comprising the amino acid sequence set forth as SEQ ID NO:10; c) an immunoglobulin V_{H} domain comprising the amino acid sequence set forth as SEQ ID NO:8 and an immunoglobulin V_{L} domain comprising the amino acid sequence set forth as SEQ ID NO:10; d) an immunoglobulin V_{H} domain comprising the hypervariable regions set forth as SEQ ID NO:1, SEQ ID NO:2, and SEQ ID NO:3; e) an immunoglobulin V_{L} domain comprising the hypervariable regions set forth as SEQ ID NO:4, SEQ ID NO:5 and SEQ ID NO:6; f) an immunoglobulin V_{H} domain comprising the hypervariable regions set forth as SEQ ID NO:11, SEQ ID NO:12 and SEQ ID NO:13; g) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or h) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

For ease of reference the amino acid sequences of the hypervariable regions of the secukinumab monoclonal antibody, based on the Kabat definition and as determined by the X-ray analysis and using the approach of Chothia and coworkers, is provided in Table 3, below.

TABLE 3
Amino acid sequences of the
hypervariable regions of the
secukinumab monoclonal antibodies.
Light-Chain

| | | |
|---|---|---|
| CDR1′ | Kabat | R-A-S-Q-S-V-S-S-S-Y-L-A |
| | | (SEQ ID NO: 4) |
| | Chothia | R-A-S-Q-S-V-S-S-S-Y-L-A |
| | | (SEQ ID NO: 4) |
| CDR2′ | Kabat | G-A-S-S-R-A-T |
| | | (SEQ ID NO: 5) |
| | Chothia | G-A-S-S-R-A-T |
| | | (SEQ ID NO: 5) |
| CDR2′ | Kabat | Q-Q-Y-G-S-S-P-C-T |
| | | (SEQ ID NO: 6) |
| | Chothia | Q-Q-Y-G-S-S-P-C-T |
| | | (SEQ ID NO: 6) |

Heavy-Chain

| | | |
|---|---|---|
| CDR1 | Kabat | N-Y-W-M-N |
| | | (SEQ ID NO: 1) |
| CDR1-x | Chothia | G-F-T-F-S-N-Y-W-M-N |
| | | (SEQ ID NO: 11) |
| CDR2 | Kabat | A-I-N-Q-D-G-S-E-K-Y-Y-V-G-S-V-K-G |
| | | (SEQ ID NO: 2) |
| CDR2-x | Chothia | A-I-N-Q-D-G-S-E-K-Y-Y |
| | | (SEQ ID NO: 12) |
| CDR3 | Kabat | D-Y-Y-D-I-L-T-D-Y-Y-I-H-Y-W-Y-F-D-L |
| | | (SEQ ID NO: 3) |
| CDR3-x | Chothia | C-V-R-D-Y-Y-D-I-L-T-D-Y-Y-I-H-Y- |
| | | W-Y-F-D-L-W-G (SEQ ID NO: 13) |

In preferred embodiments, the constant region domains preferably also comprise suitable human constant region domains, for instance as described in “Sequences of Proteins of Immunological Interest”, Kabat E. A. et al, US Department of Health and Human Services, Public Health Service, National Institute of Health. The DNA encoding the VL of secukinumab is set forth in SEQ ID NO:9. The DNA encoding the VH of secukinumab is set forth in SEQ ID NO:7.

In some embodiments, the IL-17 antagonist, e.g., IL-17 binding molecule (e.g., IL-17 antibody or antigen binding fragment thereof, e.g., secukinumab) comprises the three CDRs of SEQ ID NO:10. In other embodiments, the IL-17 antagonist comprises the three CDRs of SEQ ID NO:8. In other embodiments, the IL-17 antagonist comprises the three CDRs of SEQ ID NO:10 and the three CDRs of SEQ ID NO:8. CDRs of SEQ ID NO:8 and SEQ ID NO:10, according to both the Chothia and Kabat definition, may be found in Table 3.

In some embodiments, the IL-17 antagonist, e.g., IL-17 binding molecule (e.g., IL-17 antibody or antigen binding fragment thereof, e.g., secukinumab) comprises the light chain of SEQ ID NO:15. In other embodiments, the IL-17 antagonist comprises the heavy chain of SEQ ID NO:17. In other embodiments, the IL-17 antagonist comprises the light chain of SEQ ID NO:15 and the heavy domain of SEQ ID NO:17. In some embodiments, the IL-17 antagonist comprises the three CDRs of SEQ ID NO:15. In other embodiments, the IL-17 antagonist comprises the three CDRs of SEQ ID NO:17. In other embodiments, the IL-17 antagonist comprises the three CDRs of SEQ ID NO:15 and the three CDRs of SEQ ID NO:17. CDRs of SEQ ID NO:15 and SEQ ID NO:17, according to both the Chothia and Kabat definition, may be found in Table 3. The DNA encoding the light chain of secukinumab is set forth as SEQ ID NO:14. The DNA encoding the heavy chain of secukinumab is set forth as SEQ ID NO:16.

Hypervariable regions may be associated with any kind of framework regions, though preferably are of human origin. Suitable framework regions are described in Kabat E. A. et al, ibid. The preferred heavy chain framework is a human heavy chain framework, for instance that of the secukinumab antibody. It consists in sequence, e.g. of FR1 (amino acid 1 to 30 of SEQ ID NO:8), FR2 (amino acid 36 to 49 of SEQ ID NO:8), FR3 (amino acid 67 to 98 of SEQ ID NO:8) and FR4 (amino acid 117 to 127 of SEQ ID NO:8) regions. Taking into consideration the determined hypervariable regions of secukinumab by X-ray analysis, another preferred heavy chain framework consists in sequence of FR1-x (amino acid 1 to 25 of SEQ ID NO:8), FR2-x (amino acid 36 to 49 of SEQ ID NO:8), FR3-x (amino acid 61 to 95 of SEQ ID NO:8) and FR4 (amino acid 119 to 127 of SEQ ID NO:8) regions. In a similar manner, the light chain framework consists, in sequence, of FR1′ (amino acid 1 to 23 of SEQ ID NO:10), FR2′ (amino acid 36 to 50 of SEQ ID NO:10), FR3′ (amino acid 58 to 89 of SEQ ID NO:10) and FR4′ (amino acid 99 to 109 of SEQ ID NO:10) regions.

In one embodiment, the IL-17 antagonist, e.g., IL-17 binding molecule (e.g., IL-17 antibody or antigen binding fragment thereof, e.g., secukinumab) is selected from a human anti IL-17 antibody which comprises at least: a) an immunoglobulin heavy chain or fragment thereof which comprises a variable domain comprising in sequence the hypervariable regions CDR1, CDR2 and CDR3 and the constant part or fragment thereof of a human heavy chain; said CDR1 having the amino acid sequence SEQ ID NO:1, said CDR2 having the amino acid sequence SEQ ID NO:2, and said CDR3 having the amino acid sequence SEQ ID NO:3; and b) an immunoglobulin light chain or fragment thereof which comprises a variable domain comprising in sequence the hypervariable regions CDR1′, CDR2′, and CDR3′ and the constant part or fragment thereof of a human light chain, said CDR1′ having the amino acid sequence SEQ ID NO: 4, said CDR2′ having the amino acid sequence SEQ ID NO:5, and said CDR3′ having the amino acid sequence SEQ ID NO:6.

In one embodiment, the IL-17 antagonist, e.g., IL-17 binding molecule (e.g., IL-17 antibody or antigen binding fragment thereof, e.g., secukinumab) is selected from a single chain binding molecule which comprises an antigen binding site comprising: a) a first domain comprising in sequence the hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; and b) a second domain comprising the hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6; and c) a peptide linker which is bound either to the N-terminal extremity of the first domain and to the C-terminal extremity of the second domain or to the C-terminal extremity of the first domain and to the N-terminal extremity of the second domain.

Alternatively, an IL-17 antagonist, e.g., IL-17 binding molecule (e.g., IL-17 antibody or antigen binding fragment thereof) for use in the disclosed methods may comprise a derivative of the IL-17 binding molecules set forth herein by sequence (e.g., a pegylated version of secukinumab). Alternatively, the V_{H} or V_{L} domain of an IL-17 antagonist, e.g., IL-17 binding molecule (e.g., IL-17 antibody or antigen binding fragment thereof) for use in the disclosed methods may have V_{H} or V_{L} domains that are substantially identical to the the V_{H} or V_{L} domains set forth herein (e.g., those set forth in SEQ ID NO:8 and 10). A human IL-17 antibody disclosed herein may comprise a heavy chain that is substantially identical to that set forth as SEQ ID NO:17 and/or a light chain that is substantially identical to that set forth as SEQ ID NO:15. A human IL-17 antibody disclosed herein may comprise a heavy chain that comprises SEQ ID NO:17 and a light chain that comprises SEQ ID NO:15. A human IL-17 antibody disclosed herein may comprise: a) one heavy chain which comprises a variable domain having an amino acid sequence substantially identical to that shown in SEQ ID NO:8 and the constant part of a human heavy chain; and b) one light chain which comprises a variable domain having an amino acid sequence substantially identical to that shown in SEQ ID NO:10 and the constant part of a human light chain. Alternatively, an IL-17 antagonist, e.g., IL-17 binding molecule (e.g., IL-17 antibody or antigen binding fragment thereof) for use in the disclosed methods may be an amino acid sequence variant of the reference IL-17 binding molecules set forth herein. In all such cases of derivative and variants, the IL-17 antagonist is capable of inhibiting the activity of about 1 nM (=30 ng/ml) human IL-17 at a concentration of about 50 nM or less, about 20 nM or less, about 10 nM or less, about 5 nM or less, about 2 nM or less, or more preferably of about 1 nM or less of said molecule by 50%, said inhibitory activity being measured on IL-6 production induced by hu-IL-17 in human dermal fibroblasts.

The inhibition of the binding of IL-17 to its receptor may be conveniently tested in various assays including such assays as described in WO 2006/013107. By the term “to the same extent” is meant that the reference and the derivative molecules exhibit, on a statistical basis, essentially identical IL-17 inhibitory activity in one of the assays referred to herein (see Example 1 of WO 2006/013107). For example, the IL-17 binding molecules disclosed herein typically have IC₅₀s for the inhibition of human IL-17 on IL-6 production induced by human IL-17 in human dermal fibroblasts which are below about 10 nM, more preferably about 9, 8, 7, 6, 5, 4, 3, 2, or about 1 nM of that of, preferably substantially the same as, the IC₅₀ of the corresponding reference molecule when assayed as described in Example 1 of WO 2006/013107. Alternatively, the assay used may be an assay of competitive inhibition of binding of IL-17 by soluble IL-17 receptors (e.g. the human IL-17 R/Fc constructs of Example 1 of WO 2006/013107) and the IL-17 antagonists of the disclosure.

The disclosure also includes IL-17 antagonists, e.g., IL-17 binding molecules (e.g., IL-17 antibody or antigen binding fragment thereof, e.g., secukinumab) in which one or more of the amino acid residues of CDR1, CDR2, CDR3, CDR1-x, CDR2-x, CDR3-x, CDR1′, CDR2′ or CDR3′ or the frameworks, typically only a few (e.g., 1-4), are changed; for instance by mutation, e.g., site directed mutagenesis of the corresponding DNA sequences. The disclosure includes the DNA sequences coding for such changed IL-17 antagonists. In particular the disclosure includes IL-17 binding molecules in which one or more residues of CDR1′ or CDR2′ have been changed from the residues shown in SEQ ID NO:4 (for CDR1′) and SEQ ID NO:5 (for CDR2′).

The disclosure also includes IL-17 antagonists, e.g., IL-17 binding molecules (e.g., IL-17 antibody or antigen binding fragment thereof, e.g., secukinumab) that have binding specificity for human IL-17, in particular IL-17 antibodies capable of inhibiting the binding of IL-17 to its receptor and IL-17 antibodies capable of inhibiting the activity of 1 nM (=30 ng/ml) human IL-17 at a concentration of about 50 nM or less, about 20 nM or less, about 10 nM or less, about 5 nM or less, about 2 nM or less, or more preferably of about 1 nM or less of said molecule by 50% (said inhibitory activity being measured on IL-6 production induced by hu-IL-17 in human dermal fibroblasts).

In some embodiments, the IL-17 antagonist, e.g., IL-17 antibody, e.g., secukinumab, binds to an epitope of mature human IL-17 comprising Leu74, Tyr85, His86, Met87, Asn88, Val124, Thr125, Pro126, Ile127, Val128, His129. In some embodiments, the IL-17 antibody, e.g., secukinumab, binds to an epitope of mature human IL-17 comprising Tyr43, Tyr44, Arg46, Ala79, Asp80. In some embodiments, the IL-17 antibody, e.g., secukinumab, binds to an epitope of an IL-17 homodimer having two mature human IL-17 chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain. The residue numbering scheme used to define these epitopes is based on residue one being the first amino acid of the mature protein (ie., IL-17A lacking the 23 amino acid N-terminal signal peptide and beginning with Glycine). The sequence for immature IL-17A is set forth in the Swiss-Prot entry Q16552. In some embodiments, the IL-17 antibody has a K_{D} of about 100-200 pM. In some embodiments, the IL-17 antibody has an IC₅₀ of about 0.4 nM for in vitro neutralization of the biological activity of about 0.67 nM human IL-17A. In some embodiments, the absolute bioavailability of subcutaneously (s.c.) administered IL-17 antibody has a range of about 60- about 80%, e.g., about 76%. In some embodiments, the IL-17 antagonist, e.g., an IL-17 binding molecule (e.g., an IL-17 antibody, such as secukinumab) or an IL-17 receptor binding molecule (e.g., an IL-17 receptor antibody) has an elimination half-life of about 4 weeks (e.g., about 23 to about 35 days, about 23 to about 30 days, e.g., about 30 days). In some embodiments, the IL-17 antagonist, e.g., an IL-17 binding molecule (e.g., an IL-17 antibody, such as secukinumab) or an IL-17 receptor binding molecule (e.g., an IL-17 receptor antibody) has a Tₘₐₓ of about 7-8 days.

Particularly preferred IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof) for use in the disclosed methods, uses, kits, etc. are human antibodies, especially secukinumab as described in Examples 1 and 2 of WO 2006/013107. Secukinumab is a recombinant high-affinity, fully human monoclonal anti-human interleukin-17A (IL-17A, IL-17) antibody of the IgG1/kappa isotype that is currently in clinical trials for the treatment of immune-mediated inflammatory conditions. Secukinumab (see, e.g., WO2006/013107 and WO2007/117749) has a very high affinity for IL-17, i.e., a K_{D} of about 100-200 pM and an IC₅₀ for in vitro neutralization of the biological activity of about 0.67 nM human IL-17A of about 0.4 nM. Thus, secukinumab inhibits antigen at a molar ratio of about 1:1. This high binding affinity makes the secukinumab antibody particularly suitable for therapeutic applications. Furthermore, it has been determined that secukinumab has a very long half life, i.e., about 4 weeks, which allows for prolonged periods between administration, an exceptional property when treating chronic life-long disorders, such as PsA.

Other preferred IL-17 antibodies for use in the disclosed methods, kits and uses are those set forth in U.S. Pat. Nos. 8,057,794; 8,003,099; 8,110,191; and 7,838,638 and US Published Patent Application Nos: 20120034656 and 20110027290.

### Diagnostic Methods and Methods of Producing a Transmittable Form of Information

The disclosed methods are useful for the treatment, prevention, or amelioration of PsA, as well as predicting the likelihood of a PsA patient's response to treatment with an IL-17 antagonist, e.g., secukinumab. These methods employ, inter alia, determining whether a patient has the presence (or absence) of a PsA non-response allele or a PsA response allele in a sample from the patient.

A biological sample from the patient may be assayed for the presence or absence of a PsA non-response allele or a PsA response allele (and/or a candidate PsA response marker) by any applicable conventional means, e.g., Western blot, immunohistochemistry, Northern blot, ELISA, mass spectrometry (eg, SELDI-TOF, LC, nano-LC, UV-MALDI), immunodepletion, etc. The invention is not limited by the types of assays used to assess the presence or absence of a PsA non-response allele or a PsA response allele (and/or a candidate PsA response marker) in a biological sample from a patient. Indeed, any well-known assay that can be employed to determine the genotypic status of a patient or a level of mRNA or protein (if applicable) in a biological sample from a patient can be employed for the purposes of the present invention.

The invention is also not limited by the source of the biological sample, as numerous biological samples may be used to identify the presence or absence of a PsA non-response allele or a PsA response allele (and/or a candidate PsA response marker), e.g., blood, synovial fluid, buffy coat, serum, plasma, lymph, feces, urine, tear, saliva, cerebrospinal fluid, buccal swabs, sputum, or tissue. The invention is also not limited by the source within the biological sample used to identify the presence or absence of a PsA non-response allele or a PsA response allele (and/or a candidate PsA response marker). It will be recognized that one may assay genomic DNA obtained from a biological sample to detect a PsA non-response allele or a PsA response allele, or one may assay products of a PsA non-response allele or a PsA response allele, e.g., nucleic acid products (e.g., DNA, pre-mRNA, mRNA, micro RNAs, etc.) and polypeptide products (e.g., expressed proteins) obtained from a biological sample.

As described previously, we have determined that: 1) PsA patients carrying at least one rs240993 “T” allele, which is linked to TRAF3IP2, display reduced response relative to PsA patients that do not carry at least one rs240993 “T” allele; 2) PsA patients carrying at least one HLA-DRB1*04 allele display improved response to secukinumab relative to PsA patients that do not carry at least one HLA-DRB1*04 allele; and 3) PsA patients carrying at least one TNFSF15 rs4263839 “A” allele display improved response to secukinumab relative to PsA patients that do not carry at least one rs4263839 “A” allele. Both the rs240993 SNP and the rs4263839 SNP are found in introns, such that a patient's allelic status may be determined by interrogating, e.g., pre-mRNA or genomic DNA. However, the presence or absence of an HLA-DRB1*04 allele may be determined by assaying genomic DNA, RNA and/or serological protein. Accordingly, a skilled artisan will understand that one may identify whether a subject has a PsA non-response allele or a PsA response allele (or a candidate PsA response marker) by assaying a nucleic acid product (e.g., DNA or RNA) of a PsA non-response allele or a PsA response allele, a polypeptide product of a PsA response allele (in the case of HLA-DRB1*04 allele), or an equivalent genetic marker of a PsA non-response allele or a PsA response allele. In preferred embodiments, a genomic sequence of a PsA non-response allele or a PsA response allele is analyzed to determine whether a subject has a PsA non-response allele or a PsA response allele.

The presence or absence of a PsA non-response allele or a PsA response allele (or a candidate PsA response marker) may be detected by any of a variety of genotyping techniques commonly used in the art. Typically, such genotyping techniques employ one or more oligonucleotides that are complementary to a region containing, or adjacent to, the polymorphic site (e.g., SNP) of interest. The sequence of an oligonucleotide used for genotyping a particular polymorphic site of interest is typically designed based on a context sequence or a reference sequence.

Numerous methods and devices are well known to the skilled artisan to identify the presence or absence of a PsA non-response allele or a PsA response allele (or a candidate PsA response marker). DNA (genomic and cDNA) for SNP detection can be prepared from a biological sample by methods well known in the art, e.g., phenol/chloroform extraction, PUREGENE DNA® purification system from GentAS Systems (Qiagen, CA). Detection of a DNA sequence may include examining the nucleotide(s) located at either the sense or the anti-sense strand within that region. The presence or absence of a PsA non-response allele in a patient may be detected from DNA (genomic or cDNA) obtained from PCR using sequence-specific probes, e.g., hydrolysis probes from Taqman, Beacons, Scorpions; or hybridization probes that detect a PsA non-response allele or a PsA response allele (or an candidate PsA response marker). For the detection of the SNP, sequence specific probes may be designed such that they specifically hybridize to the genomic DNA for the alleles of interest or, in some cases, an RNA of interest. For example, Sequence specific primers and probes for rs4263839 may be found in Zucchelli et al. (2011) Gut 60:1671-77. Other primers and probes for SNPs may be designed based on context sequences found in the NCBI SNP database available at: www.ncbi.nlm.nih.gov/snp. These probes may be labeled for direct detection or contacted by a second, detectable molecule that specifically binds to the probe. The PCR products also can be detected by DNA-binding agents. Said PCR products can then be subsequently sequenced by any DNA sequencing method available in the art. Alternatively the presence or absence of allele can be detected by sequencing using any sequencing methods such as, but not limited to, Sanger-based sequencing, pyrosequencing or next generation sequencing (Shendure J. and Ji, H., Nature Biotechnology (1998), Vol. 26, Nr 10, pages 1135-1145). Optimised allelic discrimination assays for SNPs may be purchased from Applied Biosystems (Foster City, Calif., USA).

Various well-known techniques can be applied to interrogate a particular SNP, including, e.g., hybridization-based methods, such as dynamic allele-specific hybridization (DASH) genotyping, SNP detection through molecular beacons (Abravaya K., et al. (2003) Clin Chem Lab Med. 41:468-474), Luminex xMAP technology, Illumina Golden Gate technology and commercially available high-density oligonucleotide SNP arrays (e.g., the Affymetrix Human SNP 5.0 GeneChip performs a genome-wide assay that can genotype over 500,000 human SNPs), BeadChip kits from Illumina, e.g, Human660W-Quad and Human 1.2M-Duo); enzyme-based methods, such as restriction fragment length polymorphism (RFLP), PCR-based methods (e.g., Tetra-primer ARMS-PCR), Invader assays (Olivier M. (2005) Mutat Res. 573(1-2):103-10), various primer extension assays (incorporated into detection formats, e.g., MALDI-TOF Mass spectrometry, electrophoresis, blotting, and ELISA-like methods), Taqman assays, and oligonucleotide ligase assays; and other post-amplification methods, e.g., analysis of single strand conformation polymorphism (Costabile et al. (2006) Hum. Mutat. 27(12):1163-73), temperature gradient gel electrophoresis (TGGE), denaturing high performance liquid chromatography, high-resolution melting analysis, DNA mismatch-binding protein assays (e.g., MutS protein from Thermus aquaticus binds different single nucleotide mismatches with different affinities and can be used in capillary electrophoresis to differentiate all six sets of mismatches), SNPLex® (proprietary SNP detecting system available from Applied Biosystems), capillary electrophoresis, mass spectrometry, and various sequencing methods, e.g pyrosequencing and next generation sequencing, etc. Commercial kits for SNP genotyping include, e.g., Fluidigm Dynamic Array® IFCs (Fluidigm), TaqMan® SNP Genotyping Assay (Applied Biosystems), MassARRAY® iPLEX Gold (Sequenom), Type-it Fast® SNP Probe PCR Kit (Quiagen), etc.

In some embodiments, the presence or absence of an allele or SNP in a patient is detected using a hybridization assay. In a hybridization assay, the presence or absence of the genetic marker is determined based on the ability of the nucleic acid from the sample to hybridize to a complementary nucleic acid molecule, e.g., an oligonucleotide probe. A variety of hybridization assays are available. In some, hybridization of a probe to the sequence of interest is detected directly by visualizing a bound probe, e.g., a Northern or Southern assay. In these assays, DNA (Southern) or RNA (Northern) is isolated. The DNA or RNA is then cleaved with a series of restriction enzymes that cleave infrequently in the genome and not near any of the markers being assayed. The DNA or RNA is then separated, e.g., on an agarose gel, and transferred to a membrane. A labeled probe or probes, e.g., by incorporating a radionucleotide or binding agent (e.g., SYBR® Green), is allowed to contact the membrane under low-, medium- or high-stringency conditions. Unbound probe is removed and the presence of binding is detected by visualizing the labeled probe. In some embodiments, arrays, e.g., the MassARRAY system (Sequenom, San Diego, Calif., USA) may be used to genotype a subject.

Various methods of assaying, detecting, measuring, identifying and/or determining HLA alleles and allelic regions are known in the art. HLA-typing can be undertaken at low, intermediate or high resolution. Low resolution HLA typing refers to alleles which are reported at the two-digit level (e.g., HLA-DRB1*04). Intermediate resultion HLA-typing occurs when a level of ambiguity exists, even though a patient has been typed at the four digit level. Such intermediate resolution types may result from sequence-specific PCR (SSP) based typing where testing with the initial set of PCR primers will yield a list of possible genotypes that a particular person might have (which may require further testing with additional combinations of allele-specific primers and/or cloning and sequencing of clones before an unambiguous type is achieved). However, depending on the clinical and/or research purpose of the HLA typing, additional laboratory testing can achieve high-level (i.e., four-digit) resolution.

Low resolution HLA typing can be achieved by antibody-based serological tests. Higher reslution is achieveable with molecular (DNA-based methods). Such methods of HLA-typing include, e.g., DNA amplification techniques such as PCR and variants thereof, direct sequencing, Sequence Specific Oligonucleotide (SSO) hybridization coupled with the Luminex xMAP® technology, Sequence Specific Primer (SSP) typing, Sequence Based Typing (SBT). Traditional genotyping methods (e.g., employed in HLA typing) may also be modified for use in SNP genotyping or in identifying PsA non-response alleles, PsA response alleles and certain candidate PsA response markers. Such traditional methods include, e.g., DNA amplification techniques such as PCR and variants thereof, direct sequencing, SSO hybridization coupled with the Luminex xMAP® technology, SSP typing, and SBT.

Sequence-Specific Oligonucleotide (SSO) typing uses PCR target amplification, hybridization of PCR products to a panel of immobilized sequence-specific oligonucleotides on the beads, detection of probe-bound amplified product by color formation followed by data analysis. Those skilled in the art would understand that the described Sequence-Specific Oligonucleotide (SSO) hybridization may be performed using various commercially available kits, such as those provided by One Lambda, Inc. (Canoga Park, CA) or Lifecodes HLA Typing Kits (Tepnel Life Sciences Corp.) coupled with Luminex® technology (Luminex, Corporation, TX). LABType® SSO is a reverse SSO (rSSO) DNA typing solution that uses sequence-specific oligonucleotide (SSO) probes and color-coded microspheres to identify HLA alleles. The target DNA is amplified by polymerase chain reactions (PCR) and then hybridized with the bead probe array. The assay takes place in a single well of a 96-well PCR plate; thus, 96 samples can be processed at one time.

Sequence Specific Primers (SSP) typing is a PCR based technique which uses sequence specific primers for DNA based HLA typing. The SSP method is based on the principle that only primers with completely matched sequences to the target sequences result in amplified products under controlled PCR conditions. Allele sequence-specific primer pairs are designed to selectively amplify target sequences which are specific to a single allele or group of alleles. PCR products can be visualized on agarose gel. Control primer pairs that matches non-allelic sequences present in all samples act as an internal PCR control to verify the efficiency of the PCR amplification. Those skilled in the art would understand that low, medium and high resolution genotyping with the described sequence-specific primer typing may be performed using various commercially available kits, such as the Olerup SSP™ kits (Olerup, PA) or (Invitrogen) or Allset and ™Gold DQA1 Low resolution SSP (Invitrogen).

Sequence Based Typing (SBT) is based on PCR target amplification, followed by sequencing of the PCR products and data analysis.

In some cases, RNA, e.g., mature mRNA, pre-mRNA, can also be used to determine the presence or absence alleles and SNPs. Analysis of the sequence of mRNA transcribed from a given gene can be performed using any known method in the art including, but not limited, to Northern blot analysis, nuclease protection assays (NPA), in situ hybridization, reverse transcription-polymerase chain reaction (RT-PCR), RT-PCR ELISA, TaqMan-based quantitative RT-PCR (probe-based quantitative RT-PCR) and SYBR green-based quantitative RT-PCR. In one example, detection of mRNA levels involves contacting the isolated mRNA with an oligonucleotide that can hybridize to mRNA encoded by an HLA-DRB1*04 allele. The nucleic acid probe can typically be, for example, a full-length cDNA, or a portion thereof, such as an oligonucleotide of at least 7, 15, 30, 50, or 100 nucleotides in length and sufficient to specifically hybridize under stringent conditions to the mRNA. Hybridization of an mRNA with the probe indicates that the marker in question is being expressed. In one format, the RNA is immobilized on a solid surface and contacted with a probe, for example by running the isolated RNA on an agarose gel and transferring the mRNA from the gel to a membrane, such as nitrocellulose. Amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers. PCR products can be detected by any suitable method including, but not limited to, gel electrophoresis and staining with a DNA-specific stain or hybridization to a labeled probe.

In one embodiment, the presence of an allele in the HLA-DRB1*04 allelic group in a patient is determined by measuring RNA levels using, e.g., a PCR-based assay or reverse-transcriptase PCR (RT-PCR). In yet another aspect, the quantitative RT-PCR with standardized mixtures of competitive templates can be utilized.

In some embodiments, the presence or absence of an allele in the HLA-DRB1*04 allelic group in a patient can be determined by analyzing polypeptide products of the HLA-DRB*04 alleles. Detection of polypeptide products of HLA-DRB1*04 alleles (HLA-DR4 serologic antigens) can be performed using any known method in the art including, but not limited, to immunocytochemical staining, ELISA, flow cytometry, Western blot, spectrophotometry, HPLC, and mass spectrometry. In some embodiments, serologic-based HLA typing uses antigen-specific sera to determine a patient's HLA type.

One method for detecting polypeptide products of HLA-DRB1*04 alleles in a sample is by means of a probe that is a binding protein capable of interacting specifically with a marker protein. Preferably, labeled antibodies, binding portions thereof, or other binding partners can be used. The antibodies can be monoclonal or polyclonal in origin, or may be biosynthetically produced. The binding partners may also be naturally occurring molecules or synthetically produced. The amount of complexed proteins is determined using standard protein detection methodologies described in the art. A detailed review of immunological assay design, theory and protocols can be found in numerous texts in the art, including Practical Immunology, Butt, W. R., ed., Marcel Dekker, New York, 1984. A variety of assays are available for detecting proteins with labeled antibodies. Direct labels include fluorescent or luminescent tags, metals, dyes, radionucleides, and the like, attached to the antibody. Indirect labels include various enzymes well known in the art, such as alkaline phosphatase, hydrogen peroxidase and the like. In a one-step assay, polypeptide products of HLA-DRB1*04 alleles, if present, are immobilized and incubated with a labeled antibody. The labeled antibody binds to the immobilized target molecule. After washing to remove unbound molecules, the sample is assayed for the label.

The use of immobilized antibodies specific for the proteins or polypeptides is also contemplated by the present disclosure. The antibodies can be immobilized onto a variety of solid supports, such as magnetic or chromatographic matrix particles, the surface of an assay place (such as microtiter wells), pieces of a solid substrate material (such as plastic, nylon, paper), and the like. An assay strip can be prepared by coating the antibody or a plurality of antibodies in an array on solid support. This strip can then be dipped into the test sample and then processed quickly through washes and detection steps to generate a measurable signal, such as a colored spot.

In a two-step assay, immobilized polypeptide products of HLA-DRB1*04 alleles may be incubated with an unlabeled antibody. The unlabeled antibody complex, if present, is then bound to a second, labeled antibody that is specific for the unlabeled antibody. The sample is washed and assayed for the presence of the label. The choice of marker used to label the antibodies will vary depending upon the application. However, the choice of the marker is readily determinable to one skilled in the art. The antibodies may be labeled with a radioactive atom, an enzyme, a chromophoric or fluorescent moiety, or a colorimetric tag. The choice of tagging label also will depend on the detection limitations desired. Enzyme assays (ELISAs) typically allow detection of a colored product formed by interaction of the enzyme-tagged complex with an enzyme substrate. Some examples of radioactive atoms include ³²P, ¹²⁵I, ³H, and ¹⁴P. Some examples of enzymes include horseradish peroxidase, alkaline phosphatase, beta-galactosidase, and glucose-6-phosphate dehydrogenase. Some examples of chromophoric moieties include fluorescein and rhodamine. The antibodies may be conjugated to these labels by methods known in the art. For example, enzymes and chromophoric molecules may be conjugated to the antibodies by means of coupling agents, such as dialdehydes, carbodiimides, dimaleimides, and the like. Alternatively, conjugation may occur through a ligand-receptor pair. Some suitable ligand-receptor pairs include, for example, biotin-avidin or -streptavidin, and antibody-antigen.

In one aspect, the present disclosure contemplates the use of a sandwich technique for detecting polypeptide products of HLA-DRB1*04 alleles in biological samples. The technique requires two antibodies capable of binding the protein of interest: e.g., one immobilized onto a solid support and one free in solution, but labeled with some easily detectable chemical compound. Examples of chemical labels that may be used for the second antibody include but are not limited to radioisotopes, fluorescent compounds, and enzymes or other molecules which generate colored or electrochemically active products when exposed to a reactant or enzyme substrate. When samples containing polypeptide products of HLA-DRB1*04 alleles are placed in this system, the polypeptide products binds to both the immobilized antibody and the labeled antibody. The result is a “sandwich” immune complex on the support's surface. The complexed protein is detected by washing away nonbound sample components and excess labeled antibody, and measuring the amount of labeled antibody complexed to protein on the support's surface. The sandwich immunoassay is highly specific and very sensitive, provided that labels with good limits of detection are used.

Preferably, the presence of polypeptide products of HLA-DRB1*04 alleles in a sample is detected by radioimmunoassays or enzyme-linked immunoassays, competitive binding enzyme-linked immunoassays, dot blot, Western blot, chromatography, preferably high performance liquid chromatography (HPLC), or other assays known in the art. Specific immunological binding of the antibody to the protein or polypeptide can be detected directly or indirectly.

Dot blotting is routinely practiced by the skilled artisan to detect a desired protein using an antibody as a probe (Promega Protocols and Applications Guide, Second Edition, 1991, Page 263, Promega Corporation). Samples are applied to a membrane using a dot blot apparatus. A labeled probe is incubated with the membrane, and the presence of the protein is detected.

Western blot analysis is well known to the skilled artisan (Sambrook et al., Molecular Cloning, A Laboratory Manual, 1989, Vol. 3, Chapter 18, Cold Spring Harbor Laboratory). In Western blot, the sample is separated by SDS-PAGE. The gel is transferred to a membrane. The membrane is incubated with labeled antibody for detection of the desired protein.

Cellular typing may also be used for HLA-typing. A representative cellular assay is the mixed lymphocyte culture (MLC), which is used to determine the HLA class II types. The cellular assay is more sensitive in detecting HLA differences than serotyping. This is because minor differences unrecognized by alloantisera can stimulate T cells. This typing is designated as Dw types. Serotyped DR4 has been cellularly defined as DR4 Dw4, Dw10, Dw13, Dw14, Dw15. A review of various methods to perform HLA typing may be found as Howell et al. (2009) J Clin Pathol 2010 63: 387-390. Kits for HLA typing are available from, e.g, Biotest AG, Dreiech, German; Qiagen GmbH, Germany; One Lambda Inc., Canoga Park, CA; Tepnel Corp., Stamford, Conn.; Olerup, PA; Luminex Corporation, Austin, Tex.; Abbot Molecular, IL etc.

The assays described above involve steps such as but not limited to, immunoblotting, immunodiffusion, immunoelectrophoresis, or immunoprecipitation. In some embodiments, an automatic analyzer (e.g., a PCR machine or an automatic sequencing machine) is used to determine the presence or absence of an allele in the HLA-DRB1*04 allelic group.

A PsA non-response allele, PsA response allele or candidate PsA response marker can also be identified by detecting an equivalent genetic marker thereof, which can be, e.g., another SNP (single nucleotide polymorphism), a microsatellite marker, another allele or other kinds of genetic polymorphisms. For example, the presence of a genetic marker on the same haplotype as a PsA non-response allele, rather than a PsA non-response allele per se, may be indicative of a patient's likelihood for responding to treatment with an IL-17 antagonist. Two particular alleles at different loci on the same chromosome are said to be in linkage disequilibrium (LD) if the presence of one of the alleles at one locus tends to predict the presence of the other allele at the other locus. Such variants, which are referred to herein as linked variants, or proxy variants, may be any type of variant (e.g., a SNP, insertion or deletion) that is in high LD with the better response allele of interest. The candidate linked variant may be an allele of a polymorphism that is currently known. Other candidate linked variants may be readily identified by the skilled artisan using any technique well-known in the art for discovering polymorphisms.

The degree of LD between alleles of interest and a candidate linked variant may be determined using any LD measurement known in the art. LD patterns in genomic regions are readily determined empirically in appropriately chosen samples using various techniques known in the art for determining whether any two alleles (e.g., between nucleotides at different PSs) are in linkage disequilibrium (see, e.g., GENETIC DATA ANALYSIS II, Weir, Sineuer Associates, Inc. Publishers, Sunderland, Mass. 1996). The skilled artisan may readily select which method of determining LD will be best suited for a particular population sample size and genomic region. One of the most frequently used measures of linkage disequilibrium is r, which is calculated using the formula described by Devlin et al. (Genomics, 29(2):311-22 (1995)). “r” is the measure of how well an allele X at a first locus predicts the occurrence of an allele Y at a second locus on the same chromosome. The measure only reaches 1.0 when the prediction is perfect (e.g. X if and only if Y).

Preferably, the locus of the linked variant is in a genomic region of about 200 kilobases, more preferably 100 kilobases, more preferably about 10 kb that spans one of the polymorphic sites disclosed herein. Other linked variants are those in which the LD with the better response allele has a r2 value, as measured in a suitable reference population, of at least 0.75, more preferably at least 0.80, even more preferably at least 0.85 or at least 0.90, yet more preferably at least 0.95, and most preferably 1.0. The reference population used for this r measurement may be the general population, a population using an IL-17 antagonist, a population diagnosed with a particular condition for which the IL-17 antagonists shows efficacy (such as a PsA patient) or a population whose members are self-identified as belonging to the same ethnic group, such as Caucasian, African American, Hispanic, Latino, Native American and the like, or any combination of these categories. Preferably the reference population reflects the genetic diversity of the population of patients to be treated with the IL-17 antagonist.

One of skill in the art would recognize that the analysis of a PsA non-response allele or a PsA response allele may be carried out separately or simultaneously while analyzing other genetic sequences (e.g., a candidate PsA response marker). For example, a skilled artisan may analyze a sample for more than one PsA non-response allele, more than one PsA response allele, more than one candidate PsA response marker, and any combination thereof. Thus, in one aspect of the present disclosure, an array is provided to which probes that correspond in sequence to gene products, e.g., genomic DNA, cDNAs, mRNAs, cRNAs, polypeptides and fragments thereof, can be specifically hybridized or bound at a known position. As such, one may use such an array to concurrently analyze a biological sample from a patient for PsA non-response alleles, PsA response alleles and candidate PsA response markers.

In performing any of the methods described herein that require determining the presence of a PsA non-response allele or a PsA response allele, such determination may be made by consulting a data repository that contains sufficient information on the patient's genetic composition to determine whether the patient has the marker of interest. Preferably, the data repository lists the genotype present (or absent) in the individual. The data repository could include the individual's patient records, a medical data card, a file (e. g., a flat ASCII file) accessible by a computer or other electronic or non-electronic media on which appropriate information or genetic data can be stored. As used herein, a medical data card is a portable storage device such as a magnetic data card, a smart card, which has an on-board processing unit and which is sold by vendors such as Siemens of Munich Germany, or a flash-memory card. If the data repository is a file accessible by a computer; such files may be located on various media, including: a server, a client, a hard disk, a CD, a DVD, a personal digital assistant such as a Palm Pilot a tape, a zip disk, the computer's internal ROM (read-only-memory) or the internet or worldwide web. Other media for the storage of files accessible by a computer will be obvious to one skilled in the art.

Typically, once the presence of a PsA non-response allele or a PsA response allele is determined, physicians or genetic counselors or patients or other researchers may be informed of the result. Specifically the result can be cast in a transmittable form of information that can be communicated or transmitted to other researchers or physicians or genetic counselors or patients. Such a form can vary and can be tangible or intangible. The result with regard to the presence of a PsA non-response allele or a PsA response allele in the individual tested can be embodied in descriptive statements, diagrams, photographs, charts, images or any other visual forms. For example, images of gel electrophoresis of PCR products can be used in explaining the results. Diagrams showing where a variant occurs in an individual's allele are also useful in indicating the testing results. The statements and visual forms can be recorded on a tangible media such as papers, computer readable media such as floppy disks, compact disks, etc., or on an intangible media, e.g., an electronic media in the form of email or website on internet or intranet. In addition, the result with regard to the presence of a PsA non-response allele or a PsA response allele in the individual tested can also be recorded in a sound form and transmitted through any suitable media, e.g., analog or digital cable lines, fiber optic cables, etc., via telephone, facsimile, wireless mobile phone, internet phone and the like. All such forms (tangible and intangible) would constitute a “transmittable form of information”. Thus, the information and data on a test result can be produced anywhere in the world and transmitted to a different location. For example, when a genotyping assay is conducted offshore, the information and data on a test result may be generated and cast in a transmittable form as described above. The test result in a transmittable form thus can be imported into the U.S. Accordingly, the present disclosure also encompasses a method for producing a transmittable form of information on the presence of a PsA non-response allele or a PsA response allele in an individual. This form of information is useful for predicting the responsiveness of a patient having PsA to treatment with an IL-17 antagonist.

Disclosed herein are methods of predicting the likelihood that a patient having PsA will respond to treatment with an IL-17 antagonist, comprising assaying a biological sample from the patient for the presence of a PsA non-response allele or the presence of a PsA response allele, wherein: a) the presence of the PsA non-response allele is indicative of a decreased likelihood that the patient will respond to treatment with the IL-17 antagonist; and b) the presence of the PsA response allele is indicative of an increased likelihood that the patient will respond to treatment with the IL-17 antagonist.

In some embodiments, the method further comprises the step of obtaining the biological sample from the patient, wherein the step of obtaining is performed prior to the step of assaying.

In some embodiments of the disclosed methods, the biological sample is assayed for the presence of a PsA non-response allele and further wherein the PsA non-response allele is an rs240993 non-response allele.

In some embodiments of the disclosed methods, the biological sample is assayed for the presence of a PsA response allele and further wherein the PsA response allele is an rs4263839 response allele.

In some embodiments of the disclosed methods, the biological sample is assayed for the presence of a PsA response allele and further wherein the PsA response allele is an allele in the HLA-DRB1*04 allelic group.

In some embodiments of the above methods, the presence or absence of an allele of interest may be detected by assaying the biological sample for a nucleic acid product, a polypeptide product, or an equivalent genetic marker (as the case may be). The allele of interest may be the rs240993 non-response allele, a HLA-DRB1*04 allele, and/or the rs4263839 response allele. In some embodiments of the above methods, the biological sample is additionally assayed for the presence of at least one candidate PsA response marker (Table 2) selected from the group consisting of an IL13 SNP, an IL17A SNP, an IL23R SNP, an IL12B SNP, an TNIP1 SNP, a TNFAIP3 SNP, a STAT2 SNP, a SPATA2 SNP, an LCE3A SNP, and ERAP SNP, a TRAF3IP2 SNP, an HLA-C allele, an HLA-B allele, e.g., HLA-C*0602, rs20541, rs1974226, rs11209026, rs2082412, rs17728338, rs610604, rs2066808, rs2201841, rs495337, rs4085613, rs10484554, rs7747909, rs30187, rs27434, rs27524, rs33980500, rs12188300.

In some embodiments of the above methods, the biological sample is selected from the group consisting of synovial fluid, blood, serum, feces, plasma, urine, tear, saliva, cerebrospinal fluid, a leukocyte sample and a tissue sample. In some embodiments of the above methods, the presence (or absence) of the allele of interest is detected by a technique selected from the group consisting of Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southernblot, immunoassays, immunohistochemistry, ELISA, flow cytometry, Western blot, HPLC, and mass spectrometry. Assaying may be performed by use of an “automatic analyzer”, which is any machine that can be used to determine the presence or absence of an allele of interest. For example, a PCR machine, an automatic sequencer, a spectrometer, a densitometer, a plate reader, a scintillation counter, etc.

### Methods of Treatment and Uses of IL-17 Antagonists

The disclosed methods allow clinicians to provide a personalized therapy for AS patients, i.e., they allow determination of whether to treat the PsA patient with an IL-17 antagonist or whether to treat the PsA patient with a different PsA agent (e.g., NSAIDs, TNF alpha antagonists, DMARDS or corticosteroids). In this way, a clinician can maximize the benefit and minimize the risk of IL-17 antagonism in the entire population of patients afflicted with PsA. It will be understood that IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof) are useful for the treatment, prevention, or amelioration of PsA (e.g., signs and symptoms & structural changes, preventing further joint erosion, improving joint structure, etc.), particularly in PsA patients that do not have a PsA non-response allele or who have a PsA response allele.

The IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof), may be used in vitro, ex vivo, or incorporated into pharmaceutical compositions and administered to individuals (e.g., human patients) in vivo to treat, ameliorate, or prevent PsA, e.g., in PsA patients who do not have a PsA non-response allele or who have a PsA response allele. A pharmaceutical composition will be formulated to be compatible with its intended route of administration (e.g., oral compositions generally include an inert diluent or an edible carrier). Other nonlimiting examples of routes of administration include parenteral (e.g., intravenous), intradermal, subcutaneous, oral (e.g., inhalation), transdermal (topical), transmucosal, and rectal administration. The pharmaceutical compositions compatible with each intended route are well known in the art.

The IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof), may be used as a pharmaceutical composition when combined with a pharmaceutically acceptable carrier. Such a composition may contain, in addition to an IL-17 antagonist, carriers, various diluents, fillers, salts, buffers, stabilizers, solubilizers, and other materials well known in the art. The characteristics of the carrier will depend on the route of administration. The pharmaceutical compositions for use in the disclosed methods may also contain additional therapeutic agents for treatment of the particular targeted disorder. For example, a pharmaceutical composition may also include anti-inflammatory agents. Such additional factors and/or agents may be included in the pharmaceutical composition to produce a synergistic effect with the IL-17 binding molecules, or to minimize side effects caused by the IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof).

Pharmaceutical compositions for use in the disclosed methods may be manufactured in conventional manner. In one embodiment, the pharmaceutical composition is provided in lyophilized form. For immediate administration it is dissolved in a suitable aqueous carrier, for example sterile water for injection or sterile buffered physiological saline. If it is considered desirable to make up a solution of larger volume for administration by infusion rather than a bolus injection, may be advantageous to incorporate human serum albumin or the patient's own heparinised blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution. Other formulations comprise liquid or lyophilized formulation.

Antibodies, e.g., antibodies to IL-17, are typically formulated either in aqueous form ready for parenteral administration or as lyophilisates for reconstitution with a suitable diluent prior to administration. In some embodiments of the disclosed methods and uses, the IL-17 antagonist, e.g., IL-17 antibody, e.g., secukinumab, is formulated as a lyophilisate. Suitable lyophilisate formulations can be reconstituted in a small liquid volume (e.g., 2 ml or less) to allow subcutaneous administration and can provide solutions with low levels of antibody aggregation. The use of antibodies as the active ingredient of pharmaceuticals is now widespread, including the products HERCEPTIN™ (trastuzumab), RITUXAN™ (rituximab), SYNAGIS™ (palivizumab), etc. Techniques for purification of antibodies to a pharmaceutical grade are well known in the art. When a therapeutically effective amount of an IL-17 antagonist, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof) is administered by intravenous, cutaneous or subcutaneous injection, the IL-17 antagonist will be in the form of a pyrogen-free, parenterally acceptable solution. A pharmaceutical composition for intravenous, cutaneous, or subcutaneous injection may contain, in addition to the IL-17 antagonist, an isotonic vehicle such as sodium chloride, Ringer's, dextrose, dextrose and sodium chloride, lactated Ringer's, or other vehicle as known in the art.

The appropriate dosage will, of course, vary depending upon, for example, the particular IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof) to be employed, the host, the mode of administration and the nature and severity of the condition being treated, and on the nature of prior treatments that the patient has undergone. Ultimately, the attending health care provider will decide the amount of the IL-17 antagonist with which to treat each individual patient. In some embodiments, the attending health care provider may administer low doses of the IL-17 antagonist and observe the patient's response. In other embodiments, the initial dose(s) of IL-17 antagonist administered to a patient are high, and then are titrated downward until signs of relapse occur. Larger doses of the IL-17 antagonist may be administered until the optimal therapeutic effect is obtained for the patient, and the dosage is not generally increased further.

In practicing some of the methods of treatment or uses of the present disclosure, a therapeutically effective amount of an IL-17 antagonist, e.g., IL-17 binding molecule (e.g., IL-17 antibody or antigen binding fragment thereof, e.g., secukinumab) or IL-17 receptor binding molecule (e.g., IL-17 antibody or antigen binding fragment thereof) is administered to a patient, e.g., a mammal (e.g., a human). While it is understood that the disclosed methods provide for differential treatment of PsA patients depending on the presence (or absence) of PsA non-response alleles or PsA response alleles, this does not preclude that, if the patient is to be ultimately treated with an IL-17 antagonist, such IL-17 antagonist therapy is necessarily a monotherapy. Indeed, if a patient is selected for treatment with an IL-17 antagonist, then the IL-17 antagonist (e.g., secukinumab) may be administered in accordance with the method of the disclosure either alone or in combination with other agents and therapies for treating PsA patients, e.g., in combination with at least one additional PsA agent, such as an immunosuppressive agent, a disease-modifying anti-rheumatic drug (DMARD) (e.g., MTX), a pain-control drug (e.g., tramadol or paracetamol), a steroid (e.g., prednisone), a non-steroidal anti-inflammatory drug (NSAID), a cytokine antagonist, a bone anabolic, a bone anti-resorptive, and combinations thereof (e.g., dual and triple therapies). When coadministered with one or more additional agents, an IL-17 antagonist may be administered either simultaneously with the other agent, or sequentially. If administered sequentially, the attending physician will decide on the appropriate sequence of administering the IL-17 antagonist in combination with other agents, as well as the appropriate dosages for co-delivery.

Non-steroidal anti inflammatory drugs and pain control agents useful in combination with secukinumab for the treatment of PsA patients include, propionic acid derivative, acetic acid derivative, enolic acid derivatives, fenamic acid derivatives, Cox inhibitors, e.g., lumiracoxib, ibuprophen, fenoprofen, ketoprofen, flurbiprofen, oxaprozin, indomethacin, sulindac, etodolac, ketorolac, nabumetone, aspirin, naproxen, valdecoxib, etoricoxib, MK0966; rofecoxib, acetominophen, Celecoxib, Diclofenac, tramadol, piroxicam, meloxicam, tenoxicam, droxicam, lornoxicam, isoxicam, mefanamic acid, meclofenamic acid, flufenamic acid, tolfenamic, valdecoxib, parecoxib, etodolac, indomethacin, aspirin, ibuprophen, firocoxib. DMARDs useful in combination with an IL-17 antagonist, e.g., secukinumab, for the treatment of AS patients that do not have an PsA non-response allele include, methotrexate (MTX), antimalarial drugs (e.g., hydroxychloroquine and chloroquine), sulfasalazine, Leflunomide, azathioprine, cyclosporin, gold salts, minocycline, cyclophosphamide, D-penicillamine, minocycline, auranofin, tacrolimus, myocrisin, chlorambucil. Steroids (e.g., glucocorticoids) useful in combination with an IL-17 antagonist, e.g., secukinumab, for the treatment of a PsA patient that do not have a PsA non-response allele include, Prednisolone, Prednisone, dexamethasone, cortisol, cortisone, hydrocortisone, methylprednisolone, betamethasone, triamcinolone, beclometasome, fludrocottisone, deoxycorticosterone, aldosterone.

Biologic agents useful in combination with an IL-17 antagonist, e.g., secukinumab, for the treatment of a PsA patient include, ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simponi®; CNTO148), ANAKINAS (Kineret®), RITUXIMAB (Rituxan®; MabThera®), ABATACEPT (Orencia®), TOCILIZUMAB (RoActemAS/Actemra®), integrin antagonists (TYSABRI® (natalizumab)), IL-1 antagonists (ACZ885 (Ilaris), AnakinAS (Kineret®)), CD4 antagonists, further IL-17 antagonists (LY2439821, RG4934, AMG827, SCH900117, R05310074, MEDI-571, CAT-2200), IL-23 antagonists, IL-20 antagonists, IL-6 antagonists, TNF alpha antagonists (e.g., TNF alpha antagonists or TNF alpha receptor antagonsits, e.g., pegsunercept, etc.), BLyS antagonists (e.g., Atacicept, Benlysta®/LymphoStat-B® (belimumab)), P38 Inhibitors, CD20 antagonists (Ocrelizumab, Ofatumumab (Arzerra®)), Interferon gamma antagonists (Fontolizumab).

An IL-17 antagonist, e.g., secukinumab, is conveniently administered parenterally, intravenously, e.g., into the antecubital or other peripheral vein, intramuscularly, or subcutaneously. The duration of intravenous (i.v.) therapy using a pharmaceutical composition of the present disclosure will vary, depending on the severity of the disease being treated and the condition and personal response of each individual patient. Also contemplated is subcutaneous (s.c.) therapy using a pharmaceutical composition of the present disclosure. The health care provider will decide on the appropriate duration of i.v. or s.c. therapy and the timing of administration of the therapy, using the pharmaceutical composition of the present disclosure.

Preferred dosing and treatment regimens (including both induction and maintenance regimens) for treating PsA patients who do not have a PsA non-response allele or who have a PsA response allele are provided in PCT Application No. PCT/US2011/064307, which is incorporated by reference herein in its entirety.

It will be understood that dose escalation may be required (e.g., during the induction and/or maintenance phase) for certain patients, e.g., patients that display inadequate response to treatment with the IL-17 antagonists, e.g., IL-17 binding molecules (e.g., IL-17 antibody or antigen binding fragment thereof, e.g., secukinumab) or IL-17 receptor binding molecules (e.g., IL-17 antibody or antigen binding fragment thereof). Thus, s.c. dosages of secukinumab may be greater than about 75 mg to about 300 mg s.c., e.g., about 80 mg, about 100 mg, about 125 mg, about 175 mg, about 200 mg, about 250 mg, about 350 mg, about 400 mg, etc.; similarly, i.v. dosages may be greater than about 10 mg/kg, e.g., about 11 mg/kg, 12 mg/kg, 15 mg/kg, 20 mg/kg, 25 mg/kg, 30 mg/kg, 35 mg/kg, etc. It will also be understood that dose reduction may also be required (e.g., during the induction and/or maintenance phase) for certain patients, e.g., patients that display adverse events or an adverse response to treatment with the IL-17 antagonist (e.g., secukinumab). Thus, dosages of secukinumab may be less than about 75 mg to about 300 mg s.c., e.g., about 25 mg, about 50 mg, about 80 mg, about 100 mg, about 125 mg, about 175 mg, about 200 mg, 250 mg, etc.; similarly, i.v. dosages may be less than about 10 mg/kg, e.g., about 9 mg/kg, 8 mg/kg, 5 mg/kg, 4 mg/kg, 3 mg/kg, 2 mg/kg, 1 mg/kg etc.

Disclosed herein are methods of selectively treating a patient having PsA, comprising either: a) selectively administering a therapeutically effective amount of an IL-17 antagonist to the patient on the basis of the patient having a PsA response allele or on the basis of the patient not having a PsA non-response allele; or b) selectively administering a therapeutically effective amount of a different PsA agent to the patient on the basis of the patient not having a PsA response allele or on the basis of the patient having a PsA non-response allele.

Disclosed herein are methods of selectively treating a patient having PsA, comprising either: a) selectively administering a therapeutically effective amount of the IL-17 antagonist to the patient on the basis of the patient having an allele in the HLA-DRB1*04 allelic group; or b) selectively administering a therapeutically effective amount of the different PsA agent to the patient on the basis of the patient not having an allele in the HLA-DRB1*04 allelic group.

Disclosed herein are methods of selectively treating a patient having PsA, comprising either: a) selectively administering a therapeutically effective amount of the IL-17 antagonist to the patient on the basis of the patient having an rs4263839 response allele; or b) selectively administering a therapeutically effective amount of the different PsA agent to the patient on the basis of the patient not having an rs4263839 response allele.

Disclosed herein are methods of selectively treating a patient having PsA, comprising either: a) selectively administering a therapeutically effective amount of an IL-17 antagonist to the patient on the basis of the patient not having an rs240993 non-response allele; or b) selectively administering a therapeutically effective amount of the different PsA agent to the patient on the basis of the patient having an rs240993 non-response allele.

In some embodiments of the disclosed methods, the PsA agent is selected from the group consisting of an NSAID, a TNF alpha antagonist, sulfasalazine, methotrexate, a corticosteroid and combinations thereof.

Disclosed herein are methods of selectively treating a patient having PsA with an IL-17 antagonist, comprising: a) selecting the patient for treatment with the IL-17 antagonist on the basis of a the patient having a PsA response allele or on the basis of the patient not having a PsA non-response allele; and b) thereafter, administering a therapeutically effective amount of the IL-17 antagonist to the patient.

Disclosed herein are methods of selectively treating a patient having PsA with an IL-17 antagonist, comprising: a)assaying a biological sample from the patient for the presence or absence of a PsA response allele or a PsA non-response allele; and b)thereafter, selectively administering to the patient either: i. a therapeutically effective amount of an IL-17 antagonist to the patient on the basis of the biological sample from the patient having a PsA response allele or on the basis of the biological sample from the patient not having a PsA non-response allele; or ii. a therapeutically effective amount of a different PsA agent on the basis of the biological sample from the patient not having a PsA response allele or on the basis of the biological sample from the patient having a PsA non-response allele.

Disclosed herein are methods of selectively treating a patient having PsA with an IL-17 antagonist, comprising: a) assaying a biological sample from the patient for the presence or absence of an PsA response allele or a PsA non-response allele; b) thereafter, selecting the patient for treatment with the IL-17 antagonist on the basis of the biological sample from the patient having the PsA response allele or on the basis of the biological sample from the patient not having the PsA non-response allele; and c) thereafter, administering a therapeutically effective amount of the IL-17 antagonist to the patient.

In some embodiments of the disclosed methods, the PsA non-response allele or the PsA response allele is detected by assaying the biological sample for a nucleic acid product of the PsA non-response allele or the PsA response allele, a polypeptide product of the PsA non-response allele or the PsA response allele, or an equivalent genetic marker of the PsA non-response allele or the PsA response allele. In some embodiments of the disclosed methods, the PsA non-response allele or the PsA response allele is detected by assaying the biological sample for a genomic sequence of the PsA non-response allele or the PsA response allele.

In some embodiments of the disclosed methods, the biological sample is assayed for the presence of a PsA non-response allele and further wherein the PsA non-response allele is an rs240993 non-response allele. In some embodiments of the disclosed methods, the biological sample is assayed for the presence of a PsA response allele and further wherein the PsA response allele is an rs4263839 response allele. In some embodiments of the disclosed methods, the biological sample is assayed for the presence of a PsA response allele and further wherein the PsA response allele is an allele in the HLA-DRB1*04 allelic group.

In some embodiments of the disclosed methods, the patient has not been previously treated for PsA or is TNF alpha antagonist naive.

In some embodiments of the disclosed methods, the biological sample is additionally assayed for the presence of at least one candidate PsA response marker selected from the group consisting of HLA-C*0602, rs20541, rs1974226, rs11209026, rs2082412, rs17728338, rs610604, rs2066808, rs2201841, rs495337, rs4085613, rs10484554, rs7747909, rs30187, rs27434, rs27524, rs33980500, and rs12188300.

In some embodiments of the disclosed methods, the biological sample is selected from the group consisting of synovial fluid, blood, serum, feces, plasma, urine, tear, saliva, cerebrospinal fluid, a leukocyte sample and a tissue sample.

In some embodiments of the disclosed methods, the presence of the at least one PsA non-response allele or the presence of the PsA response allele is detected by a technique selected from the group consisting of Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southernblot, immunoassays, immunohistochemistry, ELISA, flow cytometry, Western blot, HPLC, and mass spectrometry.

In some embodiments of the disclosed methods, the step of administering comprises intravenously administering two or three doses of about 10 mg/kg of the IL-17 antagonist to said patient, each of said doses being administered every other week.

In some embodiments of the disclosed methods, the step of administering comprises subcutaneously administering the patient about 75 mg-about 300 mg of the IL-17 antagonist weekly, twice a month (every other week), monthly, every two months or every three months.

Disclosed herein are IL-17 antagonists for use in treating PsA, characterized in that a therapeutically effective amount of the IL-17 antagonist is administered to the patient on the basis of said patient having a PsA response allele or on the basis of said patient not having a PsA non-response allele.

In some embodiments of the disclosed uses, a therapeutically effective amount of the IL-17 antagonist is administered to the patient on the basis of said patient having an rs4263839 response allele.

In some embodiments of the disclosed uses, a therapeutically effective amount of the IL-17 antagonist is administered to the patient on the basis of said patient having an allele in the HLA-DRB1*04 allelic group.

In some embodiments of the disclosed uses, a therapeutically effective amount of the IL-17 antagonist is administered to the patient on the basis of said patient not having an rs240993 non-response allele.

Disclosed herein are IL-17 antagonists for use in treating PsA, characterized in that: a) the patient is selected for treatment with the IL-17 antagonist on the basis of a the patient having a PsA response allele or on the basis of the patient not having a PsA non-response allele; and b) thereafter a therapeutically effective amount of the IL-17 antagonist is administered to the patient.

Disclosed herein are IL-17 antagonists for use in treating PsA, characterized in that: a) a biological sample is assayed for the presence or absence of an PsA non-response allele or for the presence or absence of a PsA response allele; and b) a therapeutically effective amount of the IL-17 antagonist is selectively administered to the patient on the basis of the biological sample from the patient not having the PsA non-response allele or on the basis of the biological sample from the patient having the PsA response allele.

Disclosed herein are IL-17 antagonists for use in treating an PsA patient, characterized in that: a) a biological sample is assayed for the presence or absence of a PsA non-response allele or for the presence or absence of a PsA response allele; b) the patient is selected for treatment with the IL-17 antagonist on the basis of the biological sample from the patient having the PsA response allele or on the basis of the biological sample from the patient not having the PsA non-response allele; and c) a therapeutically effective amount of the IL-17 antagonist is selectively administered to the patient.

In some embodiments of the disclosed uses, the IL-17 antagonist is to be administered intravenously to a patient in need thereof PsA three doses of about 10 mg/kg, each of the three doses being delivered every other week.

In some embodiments of the disclosed uses, the IL-17 antagonist is to be administered subcutaneously to the patient PsA a dose of about 75 mg-about 300 mg weekly, twice a month (every other week), monthly, every two months or every three months.

Disclosed herein is an IL-17 antagonist for use in the manufacture of a medicament for use in treating a patient having PsA, wherein the patient is selected for treatment on the basis of not having a PsA non-response allele or wherein the patient is selected for treatment on the basis of having a PsA response allele.

Disclosed herein is an IL-17 antagonist for the manufacture of a medicament for the treatment of PsA in a patient characterized as not having a PsA non-response allele or a patient characterized as having a PsA response allele, wherein the medicament is formulated to comprise containers, each container having either: a sufficient amount of the IL-17 antagonist to allow delivery of at least about 75 mg-about 150 mg of the IL-17 antagonist per unit dose; or a sufficient amount of the IL-17 antagonist to allow delivery of at least about 10 mg of the IL-17 antagonist/kg patient weight per unit dose. Also disclosed herein is an IL-17 antagonist for the manufacture of a medicament for the treatment of PsA in a patient characterized as not having a PsA non-response allele or a patient characterized as having a PsA response allele, wherein the medicament is formulated at a dosage to allow either: intravenous delivery of about 10 mg of the IL-17 antagonist/kg patient weight per unit dose; or subcutaneous delivery of about 75 mg-about 150 mg of the IL-17 antagonist per unit dose.

Disclosed herein is an in vitro test method for selecting a patient for treatment of PsA using an IL-17 antagonist, comprising determining if the patient has no PsA non-response allele or determining if the patient has at least one PsA response allele, wherein the patient has an improved therapeutic response to the following regimen: a) administering the patient three doses of about 10 mg/kg of an IL-17 antagonist, each of said doses being delivered every other week; and a) thereafter administering the patient about 75 mg-about 300 mg of the IL-17 antagonist twice a month, monthly, every two months or every three months, beginning during week eight.

Disclosed herein is an in vitro test method for selecting a patient for treatment of PsA using an IL-17 antagonist, comprising determining if the patient has no PsA non-response allele or determining if the patient has at least one PsA response allele, wherein the patient has an improved therapeutic response to the following regimen: a) administering the patient five doses of about 75 mg-about 300 mg of an IL-17 antagonist, each of said doses being delivered weekly; and b) thereafter administering the patient about 75 mg-about 300 mg of the IL-17 antagonist twice a month, monthly, every two months or every three months, beginning during week eight.

Disclosed herein are also methods of treating a PsA patient, comprising receiving data regarding the presence of a PsA non-response allele or the presence of a PsA response allele in a biological sample obtained from said patient; and selectively administering a therapeutically effective amount of an IL-17 antagonist to the patient if the patient does not have the PsA non-response allele or administering a therapeutically effective amount of an IL-17 antagonist to the PsA patient if patient has the PsA response allele. The phrase “receiving data” is used to mean obtaining possession of information by any available means, e.g., orally, electronically (e.g., by electronic mail, encoded on diskette or other media), written, etc.

Some of the above methods further comprise the step of obtaining the biological sample from the patient prior to the assaying step.

As used herein, the phrase “container having a sufficient amount of the IL-17 antagonist to allow delivery of [a designated dose]” is used to mean that a given container (e.g., vial, pen, syringe) has disposed therein a volume of an IL-17 antagonist (e.g., as part of a pharmaceutical composition) that can be used to provide a desired dose. As an example, if a desired dose is 75 mg, then a clinician may use 3 ml from a container that contains an IL-17 antibody formulation with a concentration of 25 mg/ml, 2 ml from a container that contains an IL-17 antibody formulation with a concentration of 37.5 mg/ml, 1 ml from a container that contains an IL-17 antibody formulation with a concentration of 75 mg/ml, 0.5 ml from a container contains an IL-17 antibody formulation with a concentration of 150 mg/ml, etc. In each such case, these containers have a sufficient amount of the IL-17 antagonist to allow delivery of the desired 75 mg dose.

As used herein, the phrase “formulated at a dosage to allow [route of administration] delivery of [a designated dose]” is used to mean that a given pharmaceutical composition can be used to provide a desired dose of an IL-17 antagonist, e.g., an IL-17 antibody, e.g., secukinumab, via a designated route of administration (e.g., s.c. or i.v.). As an example, if a desired subcutaneous dose is 75 mg, then a clinician may use 2 ml of an IL-17 antibody formulation having a concentration of 37.5 mg/ml, 1 ml of an IL-17 antibody formulation having a concentration of 75 mg/ml, 0.5 ml of an IL-17 antibody formulation having a concentration of 150 mg/ml, etc. In each such case, these IL-17 antibody formulations are at a concentration high enough to allow subcutaneous delivery of the IL-17 antibody. Subcutaneous delivery typically requires delivery of volumes of less than about 2 ml, preferably a volume of about 1 ml or less.

### Kits

The invention also encompasses kits for detecting a PsA non-response allele or a PsA response allele in a biological sample (a test sample) from a patient. Such kits can be used to predict if a patient having PsA is likely to respond (or have a higher response) to treatment with an IL-17 antagonist, e.g., IL-17 binding molecule (e.g., IL-17 antibody or antigen binding fragment thereof, e.g., secukinumab) or IL-17 receptor binding molecule (e.g., IL-17 antibody or antigen binding fragment thereof). For example, the kit can comprise a probe (e.g., an oligonucleotode, antibody, labeled compound or other agent) capable of detecting the presence (or absence) of a PsA non-response allele or a PsA response allele, products of those alleles and/or an equivalent genetic marker of those alleles in a biological sample. The kit may also comprise instructions for providing a prediction of the likelihood that the patient will respond to treatment with the IL-17 antagonist, wherein the presence of a PsA non-response allele is indicative of a decreased likelihood that the patient will respond to treatment with the IL-17 antagonist and wherein the presence of a PsA response allele is indicative of an increased likelihood that the patient will respond to treatment with the IL-17 antagonist.

Probes may specifically hybridize (as the case may be) to a nucleic acid product of the PsA non-response allele or the PsA response allele, a polypeptide product of the PsA non-response allele or the PsA response allele, or a region of a nucleic acid coding for an equivalent genetic marker of the PsA non-response allele or the PsA response allele. Exemplary probes are oligonucleotides or conjugated oligonucleotides that specifically hybridizes to the rs240993 or rs4263839 polymorphic sites or that recognize the HLA-DRB1*04 allele; a PCR primer, together with another primer, for amplifying the rs240993, rs4263839 polymorphic sites or the HLA-DRB1*04 allele (e.g., from DNA, cDNA, mRNA, etc.); an antibody that is capable of differentiating between polypeptide products encoded by the disclosed alleles (e.g., an antibody that is capable of binding the HLA-DR4 serologic antigen) primer-extension oligonucleotides, allele-specific primers, a combination of allele-specific primers, allele-specific probes, and primer extension primers, etc. Optionally, the kit can contain a probe that targets an internal control allele, which can be any allele presented in the general population. Detection of an internal control allele is designed to assure the performance of the kit. The disclosed kits can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples that can be assayed and compared to the test sample contained. Each component of the kit is usually enclosed within an individual container, and all of the various containers are within a single package along with instructions for use.

Such kits may also comprise an IL-17 antagonist, e.g., IL-17 binding molecule (e.g., IL-17 antibody or antigen binding fragment thereof, e.g., secukinumab) or IL-17 receptor binding molecule (e.g., IL-17 antibody or antigen binding fragment thereof) (e.g., in liquid or lyophilized form) or a pharmaceutical composition comprising the IL-17 antagonist (described supra). Additionally, such kits may comprise means for administering the IL-17 antagonist (e.g., a syringe and vial, a prefilled syringe, a prefilled pen) and instructions for use. These kits may contain additional therapeutic agents (described supra) for treating PsA, e.g., for delivery in combination with the enclosed IL-17 antagonist, e.g., secukinumab.

The phrase “means for administering” is used to indicate any available implement for systemically administering a drug top a patient, including, but not limited to, a pre-filled syringe, a vial and syringe, an injection pen, an autoinjector, an i.v. drip and bag, a pump, etc. With such items, a patient may self-administer the drug (i.e., administer the drug on their own behalf) or a physician may administer the drug.

### General

It will be understood that, in the above-mentioned methods, therapeutic regimens, kits, uses, and pharmaceutical compositions, an artisan may analyze more than marker. For example, it is envisioned that a clinician may choose to analyze TRAF3IP2 SNPs, TNFSF15 SNPs, HLA-DRB 1*04 alleles, and combinations thereof in a single patient. In some embodiments, even further combinations of biomarkers may be analyzed, e.g., additional genetic markers (candidate PsA response markers), transcription markers (e.g., mRNA/miRNA derived form blood, PBMCs, biopsies, etc.), and protein and cellular markers (e.g., protein biomarkers in serum or feces and Th17 and Treg cells).

In some embodiments of the disclosed methods, treatments, regimens, uses and kits, the IL-17 antagonist is an IL-17 binding molecule or an IL-17 receptor binding molecule. In some embodiments of the disclosed methods, treatments, regimens, uses and kits, the IL-17 binding molecule or an IL-17 receptor binding molecule is an IL-17 binding molecule.

In some embodiments of the disclosed methods, treatments, regimens, uses and kits, the IL-17 binding molecule is selected from the group consisting of: a) an IL-17 antibody that binds to an epitope of IL-17 comprising Leu74, Tyr85, His86, Met87, Asn88, Val124, Thr125, Pro126, Ile127, Val128, His129; b) an IL-17 antibody that binds to an epitope of IL-17 comprising Tyr43, Tyr44, Arg46, Ala79, Asp80; c) an IL-17 antibody that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain; d) an IL-17 antibody that binds to an epitope of an IL-17 homodimer having two mature IL-17 protein chains, said epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain, wherein the IL-17 binding molecule has a K_{D} of about 100-200 pM, and wherein the IL-17 binding molecule has an in vivo half-life of about 23 to about 35 days; and e) an IL-17 antibody that comprises an antibody selected from the group consisting of: i) an immunoglobulin heavy chain variable domain (V_{H}) comprising the amino acid sequence set forth PsA SEQ ID NO:8; ii) an immunoglobulin light chain variable domain (V_{L}) comprising the amino acid sequence set forth PsA SEQ ID NO:10; iii) an immunoglobulin V_{H} domain comprising the amino acid sequence set forth PsA SEQ ID NO:8 and an immunoglobulin V_{L} domain comprising the amino acid sequence set forth PsA SEQ ID NO:10; iv) an immunoglobulin V_{H} domain comprising the hypervariable regions set forth PsA SEQ ID NO:1, SEQ ID NO:2, and SEQ ID NO:3; v) an immunoglobulin V_{L} domain comprising the hypervariable regions set forth PsA SEQ ID NO:4, SEQ ID NO:5 and SEQ ID NO:6; vi) an immunoglobulin V_{H} domain comprising the hypervariable regions set forth PsA SEQ ID NO:11, SEQ ID NO:12 and SEQ ID NO:13; vii) an immunoglobulin V_{H} domain comprising the hypervariable regions set forth PsA SEQ ID NO:1, SEQ ID NO:2, and SEQ ID NO:3 and an immunoglobulin V_{L} domain comprising the hypervariable regions set forth PsA SEQ ID NO:4, SEQ ID NO:5 and SEQ ID NO:6; and viii) an immunoglobulin V_{H} domain comprising the hypervariable regions set forth PsA SEQ ID NO:11, SEQ ID NO:12 and SEQ ID NO:13 and an immunoglobulin V_{L} domain comprising the hypervariable regions set forth PsA SEQ ID NO:4, SEQ ID NO:5 and SEQ ID NO:6.

In some embodiments of the disclosed methods, treatments, regimens, uses and kits, the IL-17 binding molecule is an antibody.

In some embodiments of the disclosed methods, treatments, regimens, uses and kits, the antibody is secukinumab.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed patient matter, as defined by the appended claims.

### EXAMPLES

### Example 1

### Proof of Concept PSA Trial CAIN4572206

### Example 1.1

### Study Design CAIN4572206

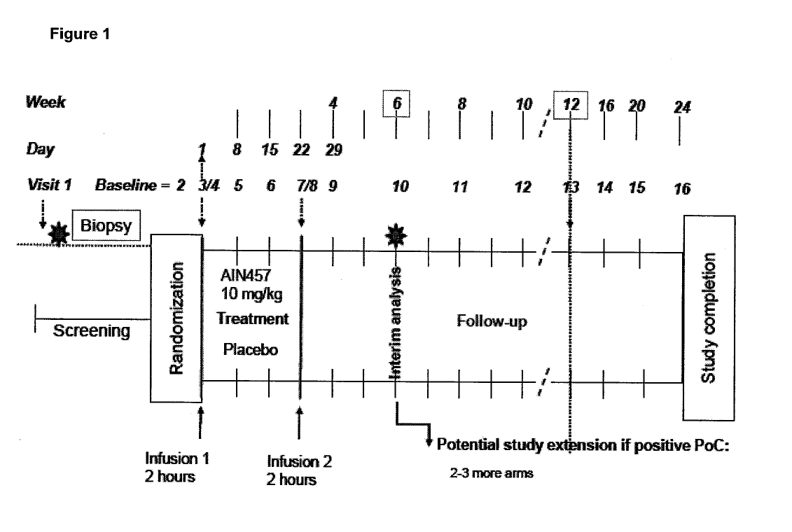

This was a randomized, double-blind, placebo controlled, multi center proof of concept study of multiple doses (2 infusions 3 weeks apart) of 10 mg/kg AIN457 for the treatment of patients with a diagnosis of active PsA based on currently advocated classification criteria for clinical trials (CASPAR). A schematic of the trial is shown in FIG. 1. Patients with moderate to severe psoriatic arthritis fulfilling the following criteria were enrolled: (i) CASPAR criteria (Taylor Wet al (2006) Arthritis Rheum 54:2665-73) for a diagnosis of psoriatic arthritis; with the modification that swelling and tenderness of at least three peripheral joints, (ii) PGA ≧40, (iii) inflammatory pain ≧40; (iv) disease is inadequately controlled on least one DMARD given for at least three months at the maximum tolerated dose (v) RF ≧100 IU AND negative CCP ELISA test. Efficacy evaluations were based on the following qualified assessment domains in accordance with the OMERACT 8 consensus: 1. peripheral joint involvement (ACR response criteria with 68/68 joint count, PsARC (Clegg et al (1996) Arthritis Rheum 39:2013-20) with DIP joints to be included in the joint count, i.e. 78/76 joint count); DAS28; 2. skin assessment (PASI score) (Feldman and Krueger (2005) Ann. Rheum. Dis. 64:ii65-ii68); 3. pain (VAS); 4. function: SF36 physical component; 5. patient global assessment by VAS (PGA); and 6. HAQ

Tender 78-Joint Count and Swollen 76-Joint Count

The distal interphalangeal joints of the feet and carpometacarpal joints of the hands were added to the usual ACR joint count of 68 tender and 66 swollen joints, to yield a 78 and 76 joint count, respectively. Thus, the joints assessed for tenderness included the distal interphalangeal, proximal interphalangeal and metacarpophalangeal joints of the hands, and metatarsophalangeal joints of the feet, the carpometacarpal and wrist joints (counted separately), the elbows, shoulders, acromioclavicular, sternoclavicular, hip, knee, talo-tibial, and mid-tarsal joints. All of these except for the hips are assessed for swelling. Joint tenderness and swelling to be graded present (1) or absent (0). The other individual elements in the ACR scoring system, VAS scores of patient pain, patient global, physician global, the Health Assessment Questionnaire (HAQ), and acute phase reactant, C-reactive protein (CRP) or erythrocyte sedimentation rate (ESR) are unchanged from the way they are used in standard trials of rheumatoid arthritis. To achieve an ACR 20, 50, or 70 response, at least 20%, 50%, or 70%, respectively, improvement in tender and swollen joint counts and three of five scores of individual elements (VAS scores of patient pain, physician and patient global assessment, a disability measure (HAQ) and an acute phase reactant (ESR or CRP)).

In addition to ACR and PsARC, DAS28 is calculated based on assessments of the following 28 joints for tenderness and swelling: metacarpophalangeal I-V (10), thumb interphalangeal (2), hand proximal interphalangeal II-V (8), wrist (2), elbow (2), shoulders (2), and knees (2).

ACR20, ACR50, ACR70 Responder Definitions

A subject is defined as an ACR20 responder if, and only if, the following three conditions hold: 1. they have a ≧20% improvement in the number of tender joints (based on 68 joints); 2. they have a ≧20% improvement in the number of swollen joints (based on 66 joints); 3. they have a ≧20% improvement in three of the following five domains:
Patient Global Assessment (measured on a VAS scale, 0-100)
Physician Global Assessment (measured on a VAS scale, 0-100)
Pain (measured on a VAS scale, 0-100)
Disability (as measured by the Health Assessment Questionnaire)
Acute phase reactant (as measured by CRP)

ACR50 and ACR70 responders are defined in a similar manner with improvements of ≧50% and ≧70% respectively.

PsARC Responder Definition

A subject is defined as a PsARC responder if, and only if, they have an improvement in two of the following four factors (with at least one factor being a joint count) and no worsening in the remaining factors
Patient global assessment (0-100 VAS scale, improvement defined as decrease of at least 20 units)
Physician global assessment (0-100 VAS scale, improvement defined as decrease of at least 20 units)
Tender 78-joint count (improvement defined as decrease of at least 30%)
Swollen 76-joint count (improvement defined as decrease of at least 30%)
   The proportion of subjects that meet each of the four responder definitions will be summarized by treatment group and time-point. Plots of these proportions over time will be presented.

DAS28 Score

The DAS28 score will be derived using the following formula: DAS28=0.56*√(tender28)+0.28√(swollen28)+0.36*loge(CRP+1)+0.014*GH+0.96, where tender28=Tender Joint Count (based on 28 joints), swollen28=Swollen Joint Count (based on 28 joints), CRP=C-reactive protein (measured in mg/L), and GH=Patients Global Assessment (measured on a VAS scale, 0-100)

Patient's Assessment of Pain Intensity

The patient's assessment of pain was performed using 100 mm VAS ranging from no pain to unbearable pain. At the investigator's site the distance in mm from the left edge of the scale was measured and the value entered on the eCRF.

Patient's Global Assessment of Disease Activity

The patient's global assessment of disease activity will be performed using 100 mm VAS ranging from not severe to very severe, after the question “In the past week how severely was your overall health affected”. At the investigator's site the distance in mm from the left edge of the scale was measured and the value entered on the eCRF.

Physician's Global Assessment of Disease Activity

The physician's global assessment of disease activity will be performed using 100 mm VAS ranging from no disease activity to maximal disease activity, after the question “Considering all the ways the disease affects your patient, draw a line on the scale for how well his or her condition is today”. To enhance objectivity, the physician must not be aware of the specific patient's global assessment of disease activity, when performing his own assessment on that patient. The investigator then measured the distance in mm from the left edge of the scale and the value entered on the eCRF.

C-Reactive Protein (CRP)

Blood for this assessment will be obtained in order to identify the presence of inflammation, to determine its severity, and to monitor response to treatment. Since the results of this test may unblind study personnel, results from the central lab will be provided for screening and baseline only. CRP results from samples collected during the treatment period were revealed following database lock only.

Erythrocyte Sedimentation Rate (ESR)

Blood will be obtained to measure ESR, which is helpful in diagnosing inflammatory diseases and is used to monitor disease activity and response to therapy. ESR was measured locally using a standard kit supplied by the central lab.

Disease Activity Score 28 (DAS28) and Patients in Remission

The DAS28 will be conducted according to the assessment schedule as described (Aletaha D, Smolen J (2005). Clin. Exp. Rheumatol; 23 (5 Suppl 39):S100-S108; Aletaha et al (2005). Arthritis Rheum.; 52 (9):2625-36). The percentage of patients in remission (DAS28≦2.6) was determined at weeks 6 and 24/end of study.

Mastricht Ankylosing Spondylitis Enthesis Score (MASES)

The Mastricht Ankylosing Spondylists Enthesis Score (MASES) (Heuft-Dorenbosch L, et al (2003) Ann Rheum Dis 62:127-32; Gladman D D (2007) Curr Rheumatol Rep 9:455-60) was developed from the Mander index, and includes assessments of 13 sites. Enthesitis sites included in the MASES index are: 1st costochondral, 7^{th} costochondral, posterior superior iliac spine, anterior superior iliac spine, iliac crest (all above assessed bilaterally), 5th lumbar spinous process, proximal Achilles (bilateral).

SPARCC (SpA Research Consortium of Canada)

SPARCC (SpA Research Consortium of Canada) (Maksymowych et al. (2003) J. Rheumatology 30:1356-63) evaluates 18 enthesis sites: medial and lateral epicondyle humerus, supraspinatus insertion, proximal Achilles, greater trochanter, medial and lateral condyl femur, insertion of plantar fascia, quadriceps insertion of patella, inferior pole of patella, tibial tubercle.

Leeds Dactylitis Instrument (LDI)

The Leeds Dactylitis Instrument (LDI) (Helliwell et al (2005). J Rheumatol 32:1745-50) basic measures the ratio of the circumference of the affected digit to the circumference of the digit on the opposite hand or foot, using a minimum difference of 10% to define a dactylitic digit. The ratio of circumference is multiplied by a tenderness score, using a modification of LDI which is a binary score (1 for tender, 0 for non-tender). If both sides are considered involved, the number will be compared to data provided in a table. This modification is referred to as LDI basic and will be applied in this study. The LDI requires a tool to measure digital circumference (available from www.rehaboutlet.com, Miami, Fla., USA).

Psoriasis Area and Severity Index (PASI)

The PASI (Feldman and Krueger (2005) Ann. Rheum. Dis. 64:ii65-ii68) assesses the extent of psoriasis on four body surface areas (head, trunk and upper and lower limbs) and the degree of plaque erythema, scaling and thickness. The PASI score accounts for the extent of body surface area affected by the erythema, scaling and thickness and the severity of these measures. The score ranges from 0 (no disease) to 72 (maximal disease).

### Example 1.2

### Secukinumab Improves Signs and Symptoms of Psoriatic Arthritis

CAIN4572206 assessed the safety and preliminary efficacy of secukinumab inhibiting Interleukin-17A, a novel target for the treatment of psoriatic arthritis (PsA). 42 patients with active PsA who fulfilled CASPAR criteria were randomized 2:1 to receive two injections of secukinumab (10 mg/kg) or placebo, given 3 weeks apart. The primary efficacy endpoint was the proportion of ACR20 responders at Week 6 in active versus placebo (one-sided p<0.01). 35 (83.3%) patients (25 on secukinumab, 10 on placebo) completed the study. 5 patients (4 secukinumab and 1 placebo) were excluded from the efficacy analysis due to protocol violations and 7 (3 secukinumab and 4 placebo) discontinued prematurely for lack of efficacy or withdrawal of consent. Demographics and baseline characteristics were balanced between groups including parameters: mean±SD SJC (secukinumab vs. placebo): 8.3±5.6 vs. 9.5±5.4; TJC 23.5±19.4 vs. 22.6±11.0; DAS28 4.8±1.2 vs. 4.8±1.2; MASES 3.0±4.1 vs. 3.4±2.3. Co-existing psoriasis, prior TNFi exposure and co-medication with DMARDS were present in 23, 11 and 21 patients on secukinumab and in 11, 5 and 10 on placebo, respectively. ACR20 responders on secukinumab vs. placebo were 39% vs. 23% (P=0.27) at Week 6, 39% vs. 15% at Week 12, 43% vs. 18% at Week 28. ACR50 and ACR70 responders on secukinumab vs. placebo were 17% vs. 8% and 9% vs. 0%, respectively at Week 6. CRP reductions at Week 6 were greater on secukinumab (median [range] at baseline vs. Week 6: 4.9 [0.3, 43.0] vs. 3.0 [0.2, 15.2]) than on placebo (6.2 [1.3, 39.7] vs. 5.0 [0.8, 29.6]).

Overall rate of adverse events (AEs) was comparable in secukinumab 26 (93%) vs. placebo 11 (79%). 7 serious AEs were reported in 4 secukinumab patients and 1 in placebo. Infections were reported in 16 (57%) patients on secukinumab and 7 (50%) on placebo. In conclusion, the primary endpoint was not met, though patients showed rapid and sustained improvements of clinical scores and CRP levels up to Week 28. The safety profile of secukinumab was favorable. These findings warrant further larger phase III clinical trials in PsA, which is ongoing as CAIN457F2306.

### Example 2

### Materials and Method for Pharmacogenetic (PG) Analysis in Psoriatic Arthritis Trial CAIN457A2206

### Example 2.1

### Samples and Processing

DNA was genotyped in 40 consenting patients who participated in the study. 27 patients who received secukinumab were used in the pharmacogenetic (PG) analysis.

Blood samples from consenting patients were collected at the individual trial sites and then shipped to Covance (Geneva, Switzerland). The genomic DNA of each patient was extracted from the blood by Covance using the PUREGENE D-50K DNA Isolation Kit (Gentra, Minneapolis, Minn., USA) and shipped to Novartis for genotyping.

14 SNPs reported to be associated with risk of PsA or related disease, as well as 5 SNPs observed to associate with secukinumab response in other indications, were genotyped. TaqMan® genotyping was performed using TaqMan Assays-by-Design and Assays-on-Demand (Applied Biosystems, Foster City, Calif.) on an ABI 7900HT sequence detection system. Up to 20 ng of genomic DNA was used in the experiment according to the manufacturer's instructions.

The HLA-C*0602 allele was also chosen for genotyping given it is the major genetic risk factor for PsA. In addition, the HLA-DRB1*04 allelic group (2-digit alleles) was included in the test because of the prior finding of association with differential response to secukinumab treatment in rheumatoid arthritis trials. All DNA samples from consenting patients in the study were tested with sequence-specific oligonucleotide hybridization (SSO) method. Briefly, SSO experiments were performed by using LABType® HD B and DRB1 Typing Test (One lambda, Inc, CA) with Luminex IS200 instrument according to manufacturer's instructions. HLA genotypes were assigned by using HLA Fusion® 2.0 software (One Lambda).

### Example 2.2

### Statistical Analysis

All variants were tested individually, i.e., only 1 variant was included in the model at a time. All HLA alleles were tested against clinical endpoints using the standard additive effect coding: individuals were coded 0, 1 or 2 for the HLA allele, depending on the number of copies of the HLA allele that an individual carries. All association tests were two-tailed, single-point tests for an additive allelic effect.

Ancestry is a common confounding factor in genetic association studies. All the 27 secukinumab-treated patients in A2206 are Caucasians. The analysis was run including only Caucasian secukinumab-treated patients (N=27).

Only secukinumab-treated patients were used for the genetic analysis in A2206 samples (N=27). The null hypothesis was that the coefficient for the genotype variable was equal to zero, and the corresponding p-value was presented. Rejecting the null hypothesis would mean concluding that genotype was a predictor of response to secukinumab as measured by the specific clinical endpoint.

Only one patient in the placebo arm reach ACR50 and only two reached ACR20. As a result the analysis was not run in the placebo arm.

All statistical tests were performed in SAS (SAS Institute Inc., Cary, N.C., USA). Efficacy variables ACR20, ACR50 and ACR70 at week 6/week 24 were analyzed separately using a logistic regression logistic regression exact tests with Lancaster's mid-p correction (SAS 9.2 PROC LOGISTIC), with the efficacy endpoint as the dependent variable, SNP or HLA alleleic group genotype (as coded above) as the independent variable (fixed effect). Efficacy variable DAS28 at week 6/week 24 were analyzed separately using an ANCOVA model (SAS 9.2 PROC GLM), with the efficacy endpoint as the dependent variable, SNP or HLA alleleic group genotype (as coded above) as the independent variable (fixed effect), and baseline DAS28 score and sex as fixed effect covariates.

### Example 3

### Results for PG Analysis in PsA Trial CAIN457A2206

### Example 3.1

### Association of Genetic Variants with Secukinumab Efficacy in Secukinumab-Treated Patients

A total of 21 genetic polymorphisms including 19 SNPs and 2 HLA allelic groups were tested for association with efficacy endpoints. 15 genetic polymorphisms were selected for analysis based on publications reporting strong evidence of association with PsA or related disease, the hypothesis being that disease SNPs may identify different disease sub-types which may lead to differential response to therapy. Six additional genetic polymophisms were selected for analysis based on their association with secukinumab in other indications.

Among the 21 variants, a SNP rs240993 T allele has the best p value, with nominal p-value of 0.015 (Table 7) for association with lower percentage ACR50 at week 24 in 27 secukinumab-treated patients. This SNP also association with ACR70 at week 24 (p-value=0.021, Table 7) and DAS28 at week 6 (p-value=0.057, Table 6). Patients having at least one rs240993 T allele display reduced response relative to PsA patients that do not carry any rs240993 T allele. The association of SNP rs240993 T allele with psoriasis disease was identified by Psoriasis Consortium & the Wellcome Trust Case Control Consortium 2 (Strange et al., 2010), which linked this SNP to the gene TRAF3IP2. TRAF3IP2 encodes ACTT, an adaptor protein essential for IL17-dependent NF-KB activation and Th17-mediated inflammatory responses (May et al (2011) Nat Immunol. 12(9):813-5). To be noted, this SNP is physically located in the intron region of REV3L gene, which is a gene immediate downstream of TRAF3IP2. As shown in FIG. 2, there is a high linkage equilibrium region across REV3L and TRAF3IP2 as shown by high R squared value and low recombination rate. A hypothesis is that rs240993 might be tagging a causal SNP in TRAF3IP2 gene.

The HLA-DRB1*04 allelic group also showed nominally significant association with ACR70 (p-value=0.016) and ACR50 (p-value=0.050) at week 24 (Table 7). The association between HLA-DRB1*04 allelic group and secukinumab response in PsA is in the same trend as observed in RA (PCT Application No. PCT/US2011/064307, which is incorporated by reference herein in its entirety). Patients carrying at least one HLA-DRB1*04 allele display improved response to secukinumab relative to PsA patients that do not carry any HLA-DRB1*04 allele.

Besides, a SNP rs4263839 A allele in an intron of the tumour necrosis factor-like ligand (TL1A) gene associates with a higher percentage ACR50 at week 6 (p-value=0.034, Table 6) and ACR70 at week 24 (p-value=0.056, Table 7). Patients carrying at least one rs4263839 A allele display improved response to secukinumab relative to PsA patients that do not carry any rs4263839 A allele. The rs4263839 G allele was identified to be associated with susceptibility to inflammatory bowel disease (Barrett et al (2008) Nat Genet. 40(8):955-62). This polymorphism also demonstrated a highly significant association with faecel calprotectin (S100A8/S100A9) response among 16 secukinumab-treated patients (p=0.00035 in permutation test after Bonferroni correction for multiple comparisons). We have previously determined that the absence of the minor allele A is associated with worsening in patients having Crohn's disease (i.e., increase in faecel calprotectin concentrations) (data not shown), which is in the same trend as observed here in PsA patients. The TL1A gene encodes a cytokine that drives pathogenic T cells in various autoimmune inflammatory processes (Bayry et al (2010) Nat Rev Rheumatol. 6(2):67-8.).

Lastly, a SNP rs7747909 in the 3′UTR region of IL17A associates with ACR70 at week 6 (p-value=0.031, Table 6). However, the association between rs7747909 and secukinumab response in PsA is in opposite direction to that observed in patients having rheumatoid arthritis (data not shown).

TABLE 6
shows the p-values from association tests for each genetic
variant against ACR20, ACR50, ACR70 and DAS28 at week 6.

| | | ACR20 OR | ACR50 OR | | ACR70 OR | DAS28 coefficient |
|---|---|---|---|---|---|---|
| Variants | Gene | (p) | (p) | | (p) | estimate (p) |
| HLA-C*0602 | HLA-C | 1.77 (0.40) | 2.58 | (0.19) | 6.67 (0.04) | −0.56 (0.14) |
| HLA-DRB1*04 | HLA-DRB1 | 4.66 (0.20) | 3.59 | (0.43) | 2.22 (0.79) | −0.57 (0.38) |
| rs20541 | IL13 | 0.66 (0.54) | 0.30 | (0.26) | NC (0.10) | 0.19 (0.71) |
| rs1974226 | IL17A | 1.53 (0.46) | 0.6 | (0.62) | 0.56 (0.56) | 0.46 (0.21) |
| rs11209026 | IL23R | 0.8 (0.88) | 1.31 | (0.61) | 4.50 (0.14) | −0.28 (0.42) |
| rs2082412 | IL12B | NC (0.10) | NC | (0.35) | NC (0.69) | 0.97 (0.17) |
| rs17728338 | TNIP1 | 1.29 (0.80) | 0.94 | (0.77) | 2.22 (0.79) | 0.32 (0.59) |
| rs610604 | TNFAIP3 | NC (0.33) | NC | (0.33) | NC (0.63) | 0.2 (0.81) |
| rs2066808 | STAT2/IL23A | 0.85 (0.89) | 0.64 | (0.63) | 1.13 (0.84) | 0.32 (0.32) |
| rs2201841 | IL23R | 0.36 (0.44) | NC | (0.17) | NC (0.34) | 1.45 (0.019) |
| rs495337 | SPATA2/ZNF313 | 1.5 (0.46) | 2.11 | (0.24) | 3.93 (0.05) | −0.22 (0.52) |
| rs4085613 | LCE3A/LCE3D | 1.00 (0.89) | 1.45 | (0.6) | 1.00 (0.85) | −0.35 (0.26) |
| rs10484554 | HLA-C/HLA-B | 1.93 (0.24) | 0.76 | (0.66) | 1.50 (0.59) | −0.09 (0.77) |
| rs7747909 | IL17A | 1.33 (0.68) | 2.10 | (0.28) | 5.06 (0.031) | −0.46 (0.14) |
| rs4263839 | TL1A | 1.53 (0.62) | 8.22 | (0.034) | 1.78 (0.49) | −0.57 (0.13) |
| rs30187 | ERAP1 | 1.21 (0.88) | (0.86) | | 1.94 (0.58) | −0.14 (0.70) |
| rs27434 | ERAP1 | 1.49 (0.57) | 4.64 | (0.13) | 4.66 (0.25) | −0.51 (0.24) |
| rs27524 | ERAP1 | 0.94 (0.88) | 0.65 | (0.63) | 1.51 (0.56) | −0.01 (0.97) |
| rs33980500 | TRAF3IP2 | 0.55 (0.50) | NC | (0.08) | NC (0.37) | 0.09 (0.88) |
| rs240993 | TRAF3IP2 | 0.30 (0.13) | 0.19 | (0.17) | 0.90 (0.81) | 0.73 (0.057) |
| rs12188300 | IL12B | 0.93 (0.86) | 0.48 | (0.55) | NC (0.25) | 0.54 (0.19) |

(NC: OR not calcualtable)

TABLE 7
shows the p-values from association tests for each genetic
variant against ACR20, ACR50, ACR70 and DAS28 at week 24.

| | | ACR20 OR | ACR50 OR | ACR70 OR | DAS28 coefficient |
|---|---|---|---|---|---|
| Variants | Gene | (p) | (p) | (p) | estimate (p) |
| HLA-C*0602 | HLA-C | 1.40 (0.63) | 1.44 (0.58) | 0.25 (0.30) | −0.4 (0.20) |
| HLA-DRB1*04 | HLA-DRB1 | 1.79 (0.48) | 6.7 (0.050) | 14.9 (0.016) | −0.46 (0.37) |
| rs20541 | IL13 | 0.54 (0.54) | 0.30 (0.26) | 0.53 (0.80) | 0.01 (0.98) |
| rs1974226 | IL17A | 1.07 (0.88) | 0.32 (0.24) | 0.5 (0.57) | 0.06 (0.85) |
| rs11209026 | IL23R | 1.39 (0.66) | 0.80 (0.87) | 0.56 (0.57) | −0.2 (0.49) |
| rs2082412 | IL12B | 2.36 (0.40) | NC (0.35) | NC (0.34) | 0.56 (0.35) |
| rs17728338 | TNIP1 | 1.09 (0.79) | 0.94 (0.77) | NC (0.35) | 0.12 (0.81) |
| rs610604 | TNFAIP3 | 2.45 (0.34) | 2.21 (0.59) | 3.15 (0.13) | −0.12 (0.77) |
| rs2066808 | STAT2/IL23A | 0.31 (0.09) | 0.70 (0.60) | 0.37 (0.30) | 0.3 (0.30) |
| rs2201841 | IL23R | 0.30 (0.46) | NC (0.17) | NC (0.35) | 0.72 (0.17) |
| rs495337 | SPATA2/ZNF313 | 1.10 (0.88) | 1.88 (0.41) | 1.27 (0.86) | −0.32 (0.27) |
| rs4085613 | LCE3A/LCE3D | 1.00 (0.89) | 1.00 (0.88) | 1.62 (0.63) | −0.25 (0.33) |
| rs10484554 | HLA-C/HLA-B | 0.96 (0.89) | 0.83 (0.8) | 0.61 (0.61) | 0 (0.99) |
| rs7747909 | IL17A | 1.52 (0.49) | 1.41 (0.65) | 0.23 (0.22) | −0.32 (0.23) |
| rs4263839 | TL1A | 2.34 (0.25) | 3.7 (0.094) | 6.53 (0.056) | −0.56 (0.071) |
| rs30187 | ERAP1 | 1.77 (0.46) | 0.63 (0.60) | 0.46 (0.55) | 0 (0.99) |
| rs27434 | ERAP1 | 1.10 (0.85) | 1.99 (0.53) | 0.80 (0.81) | −0.02 (0.96) |
| rs27524 | ERAP1 | 0.70 (0.67) | 0.65 (0.63) | 0.47 (0.37) | 0.12 (0.68) |
| rs33980500 | TRAF3IP2 | 0.66 (0.82) | NC (0.18) | NC (0.38) | 0.82 (0.11) |
| rs240993 | TRAF3IP2 | 0.47 (0.26) | 0.1 (0.015) | 0.08 (0.021) | 0.67 (0.029) |
| rs12188300 | IL12B | 0.56 (0.60) | 0.60 (0.57) | 0.89 (0.81) | 0.01 (0.97) |

(NC: OR not calcualtable)

### Example 3.2

### Effect of SNP rs240993 (Linked to TRAF3IP2) Alleles on Secukinumab Response in Secukinumab-Treated PsA Patients

Nine out of 27 secukinumab-treated patients have two copies of rs240993 major allele C. As shown in Table 8, individuals carrying two copies of rs240993 major allele C have the highest ACR20, ACR50 and ACR70 response rate at week 24, followed by heterozygous individuals (those carrying one copy of T allele and one copy of C allele). The patients carrying two copies of rs240993 minor allele T (rs240993 non-response allele) have the lowest ACR20, ACR50 and ACR70 response rate at week 24 at week 24.

TABLE 8
shows the number and percentage of secukinumab-treated patients
reaching a given endpoint (ACR20, ACR50, ACR70) at week 24, grouped
by the genotype groups of rs240993 non-response allele.

| Number/Percentage of | | | | |
|---|---|---|---|---|
| patients reaching | rs240993 | rs240993 | rs240993 | |
| endpoint in | CC | CT | TT | Overall |
| secukinumab arm | (n = 9) | (n = 16) | (n = 2) | (n = 27) |
| ACR20 (N/%) | 5 (56%) | 8 (50%) | 0 | 13 (48%) |
| ACR50 (N/%) | 5 (56%) | 2 (13%) | 0 | 7 (26%) |
| ACR70 (N/%) | 4 (44%) | 1 (6%) | 0 | 5 (19%) |

### Example 3.3

### Effect of HLA-DRB1*04 Allelic Group on Secukinumab Response in Secukinumab-Treated PsA Patients

Five out of 27 secukinumab-treated patients carry at least one copy of HLA-DRB1*04 allelic group. As shown in Table 9, individuals carrying at least one copy of HLA-DRB1*04 allele have the better ACR20, ACR50 and ACR70 response rate at week 24. The patients not carrying HLA-DRB1*04 allele have lower ACR20, ACR50 and ACR70 response rate at wk 24.

TABLE 9
shows the number and percentage of secukinumab-treated patients
reaching a given endpoint (ACR20, ACR50, ACR70) at week 24,
grouped by the genotype groups of HLA-DRB1*04 allele.

| Number/Percentage of | | | |
|---|---|---|---|
| patients reaching | HLA-DRB1*04 | HLA-DRB1*04 | |
| endpoint in | Carriers | Non-carriers | Overall |
| secukinumab arm | (n = 5) | (n = 22) | (n = 27) |
| ACR20 (N/%) | 3 (60%) | 10 (45%) | 13 (48%) |
| ACR50 (N/%) | 3 (60%) | 4 (18%) | 7 (26%) |
| ACR70 (N/%) | 3 (60%) | 2 (9%) | 5 (19%) |

### Example 3.4

### Effect of Combining SNP rs240993 (Linked to TRAF3IP2) Alleles and HLA-DRB1*04 Allelic Group on Secukinumab Response in Secukinumab-Treated PsA Patients

12 out of 27 secukinumab-treated patients carry two copies of rs240993 minor allele C or at least one copy of HLA-DRB1*04 allele. As shown in Table 10, individuals carrying two copies of rs240993 minor allele C or at least one copy of HLA-DRB1*04 allele have the better ACR20, ACR50 and ACR70 response rate at week 24. The patients not carrying rs240993 CC genotype or at least one copy of HLA-DRB1*04 allele have lower ACR20, ACR50 and ACR70 response rate at week 24.

TABLE 10
shows the number and percentage of secukinumab-treated
patients reaching a given endpoint (ACR20, ACR50,
ACR70) at week 24, grouped by the combiend genotype
groups of rs240993 T and HLA-DRB1*04.

| Number/Percentage of | rs240993 CC/ | rs240993 CC/ | |
|---|---|---|---|
| patients reaching | HLA-DRB1*04 | HLA-DRB1*04 | |
| endpoint in | Carriers | Non-carriers | Overall |
| secukinumab arm | (n = 12) | (n = 15) | (n = 27) |
| ACR20 (N/%) | 6 (50%) | 7 (47%) | 13 (48%) |
| ACR50 (N/%) | 6 (50%) | 1 (7%) | 7 (26%) |
| ACR70 (N/%) | 5 (42%) | 0 (0%) | 5 (19%) |

### Example 3.5

### Effect of TL1A SNP rs4263839 Alleles on Secukinumab Response in Secukinumab-Treated PsA Patients

14 out of 27 secukinumab-treated patients carry at least one copy of rs4263839 minor allele A. As shown in Table 11, individuals carrying two copies of rs4263839 minor allele A (rs4263839 response allele) have the highest ACR20, ACR50 and ACR70 response rate at week 24, followed by heterozygous individuals (those carrying one copy of G allele and one copy of A allele). The patients not carrying rs4263839 minor allele A have the lowest ACR20, ACR50 and ACR70 response rate at week 24.

TABLE 11
shows the number and percentage of secukinumab-treated patients
reaching a given endpoint (ACR20, ACR50, ACR70) at week 24,
grouped by the genotype groups of rs4263839 risk allele.

| Number/Percentage of | | | | |
|---|---|---|---|---|
| patients reaching | rs4263839 | rs4263839 | rs4263839 | |
| endpoint in | GG | GA | AA | Overall |
| secukinumab arm | (n = 13) | (n = 13) | (n = 1) | (n = 27) |
| ACR20 (N/%) | 5 (38%) | 7 (54%) | 1 (100%) | 13 (48%) |
| ACR50 (N/%) | 2 (15%) | 4 (31%) | 1 (100%) | 7 (26%) |
| ACR70 (N/%) | 1 (8%) | 3 (23%) | 1 (100%) | 5 (19%) |

### Example 4

### Conclusion for PGx and in PsA Trial CAIN4572206

We have shown that PsA patients carrying at least one rs240993 T allele display reduced response to secukinumab relative to PsA patients that do not carry at least one rs240993 T allele, that PsA patients carrying at least one HLA-DRB1*04 allele display improved response to secukinumab relative to PsA patients that do not carry at least one HLA-DRB1*04 allele, and that PsA patients carrying at least one rs4263839 A allele display improved response to secukinumab relative to PsA patients that do not carry at least one rs4263839 A allele. These pharmacogenomic findings could not have been predicted based solely on the fact that certain SNPs may be associated with an increased likelihood of a patient developing the PsA disease. For example, as shown in Tables 6 and 7, various other SNPs associated with the PsA disease did not predict PsA patient response to IL-17 antagonism with secukinumab—including rs12188300, rs33980500, rs20541, rs2066808, etc. As a further example of the lack of unpredictability, it is well known that HLA-DRB 1 alleles encoding the shared epitope (SE) confer higher risk for rheumatoid arthritis (RA) development (Gonzalez-Gay et al. (2002) Sem. Arthritis. Rheum. 31:355-60; Fries et al. (2002) Arthritis and Rheumatism 46:2320-29; van der Helm-van Mil et al. (2006) Arthritis and Rheum. 54:1117-21). However, it is generally accepted that carriage of the SE does not predict whether an RA patient will respond to treatment with a TNF alpha antagonist, such as etanercept and infliximab (Emery and Dorner (2011) Ann. Rhem. Dis. 70:2063-2070; Potter et al. (2009) Ann. Rheum. Dis. 68:69-74), even though the SE can be used to predict an increased likelihood that a patient will respond favorably to treatment with secukinumab (PCT Application No. PCT/US2011/064307, which is incorporated by reference herein in its entirety). As such, one cannot predict how a patient will respond to a drug based solely based on whether that patient carries an allele associated with a particular disease.

1-38. (canceled)

39. A method of selectively treating a patient having psoriatic arthritis (PsA), comprising either:
a) selectively administering a therapeutically effective amount of an IL-17 antagonist to the patient on the basis of the patient having been determined to have an allele in the HLA-DRB1*04 allelic group, having been determined to have an rs4263839 response allele, or having been determined to not have an rs240993 non-response allele; or
b) selectively administering a therapeutically effective amount of a different PsA agent to the patient on the basis of the patient having been determined to have an rs240993 non-response allele, having been determined to not have an allele in the HLA-DRB1*04 allelic group, or having been determined to not have an rs4263839 response allele.

40. The method according to claim 39, wherein the different PsA agent is selected from the group consisting of an NSAID, a TNF alpha antagonist, sulfasalazine, methotrexate, a corticosteroid and combinations thereof.

41. A method of selectively treating a patient having PsA with an IL-17 antagonist, comprising:
a) selecting the patient for treatment with the IL-17 antagonist on the basis of a the patient having been determined to have an allele in the HLA-DRB1*04 allelic group, having been determined to have an rs4263839 response allele, or having been determined to not have an rs240993 non-response allele; and
b) thereafter, selectively administering a therapeutically effective amount of the IL-17 antagonist to the patient.

42. The method according to claim 41, further comprising assaying a biological sample from the patient for the presence of an allele in the HLA-DRB1*04 allelic group, an rs4263839 response allele, or an rs240993 non-response allele, wherein the step of assaying is performed prior to the step of selecting.

43. The method according to claim 42, wherein the step of assaying comprises assaying the biological sample for a nucleic acid product of an allele in the HLA-DRB1*04 allelic group, an rs4263839 response allele, or an rs240993 non-response allele; a polypeptide product of an allele in the HLA-DRB1*04 allelic group, an rs4263839 response allele, or an rs240993 non-response allele; or an equivalent genetic marker of an allele in the HLA-DRB1*04 allelic group, an rs4263839 response allele, or an rs240993 non-response allele.

44. The method according to claim 43, wherein the step of assaying comprises assaying the biological sample for a genomic sequence of an allele in the HLA-DRB1*04 allelic group, an rs4263839 response allele, or an rs240993 non-response allele.

45. The method according to claim 41, wherein the patient is selected for treatment on the basis of the patient having been determined not to have an rs240993 non-response allele.

46. The method according to any one of claim 41, wherein the patient is selected for treatment on the basis of the patient having been determined to have an rs4263839 response allele.

47. The method according to any one of claim 41, wherein the patient is selected for treatment on the basis of the patient having been determined to have an allele in the HLA-DRB1*04 allelic group.

48. The method according to claim 42, wherein the biological sample is additionally assayed for the presence of at least one candidate PsA response marker selected from the group consisting of HLA-C*0602, rs20541, rs1974226, rs11209026, rs2082412, rs17728338, rs610604, rs2066808, rs2201841, rs495337, rs4085613, rs10484554, rs7747909, rs30187, rs27434, rs27524, rs33980500, and rs12188300.

49. The method according to claim 42, wherein the biological sample is selected from the group consisting of synovial fluid, blood, serum, feces, plasma, urine, tear, saliva, cerebrospinal fluid, a leukocyte sample and a tissue sample.

50. The method according claim 42, wherein the biological sample is assayed using a technique selected from the group consisting of Northern blot analysis, polymerase chain reaction (PCR), reverse transcription-polymerase chain reaction (RT-PCR), TaqMan-based assays, direct sequencing, dynamic allele-specific hybridization, high-density oligonucleotide SNP arrays, restriction fragment length polymorphism (RFLP) assays, primer extension assays, oligonucleotide ligase assays, analysis of single strand conformation polymorphism, temperature gradient gel electrophoresis (TGGE), denaturing high performance liquid chromatography, high-resolution melting analysis, DNA mismatch-binding protein assays, SNPLex®, capillary electrophoresis, Southernblot, immunoassays, immunohistochemistry, ELISA, flow cytometry, Western blot, HPLC, and mass spectrometry.

51. The method according to claim 41, wherein the step of selectively administering comprises:
a) intravenously administering three doses of about 10 mg/kg of the IL-17 antagonist to the patient, each of the doses being administered every other week, and thereafter subcutaneously administering about 75 mg-about 300 mg of the IL-17 antagonist to the patient monthly; or
b) subcutaneously administering five doses of about 75 mg-about 300 mg of the IL-17 antagonist to the patient, each of the five doses being administered weekly, and thereafter subcutaneously administering about 75 mg-about 300 mg of the IL-17 antagonist to the patient monthly.

52. A method of predicting the likelihood that a patient having PsA will respond to treatment with an IL-17 antagonist, comprising assaying a biological sample from the patient for the presence of an allele in the HLA-DRB1*04 allelic group, an rs4263839 response allele, or an rs240993 non-response allele, wherein:
a) the presence of an rs240993 non-response allele is indicative of a decreased likelihood that the patient will respond to treatment with the IL-17 antagonist; and
b) the presence of an allele in the HLA-DRB1*04 allelic group or an rs4263839 response allele is indicative of an increased likelihood that the patient will respond to treatment with the IL-17 antagonist.

53. The method according to claim 52, further comprising the step of obtaining the biological sample from the patient, wherein the step of obtaining is performed prior to the step of assaying.

54. The method according to claim 52, wherein the biological sample is assayed for the presence of an rs240993 non-response allele.

55. The method according to claim 52, wherein the biological sample is assayed for the presence of an rs4263839 response allele.

56. The method according to claim 52, wherein the biological sample is assayed for the presence of an allele in the HLA-DRB1*04 allelic group.

57. The method according claim 41, wherein the IL-17 antagonist is selected from the group consisting of:
a) an IL-17 antibody that binds to an epitope of IL-17 comprising Leu74, Tyr85, His86, Met87, Asn88, Val124, Thr125, Pro126, Ile127, Val128, His129;
b) an IL-17 antibody that binds to an epitope of IL-17 comprising Tyr43, Tyr44, Arg46, Ala79, Asp80:
c) an IL-17 antibody that binds to an epitope of an IL-17 homodimer having two mature IL-17 protein chains, the epitope comprising Leu74, Tyr85, His86, Met87, Asn88, Val124, Thr125, Pro126, Ile127, Val128, His129 on one chain and Tyr43, Tyr44, Arg46, Ala79, Asp80 on the other chain;
d) an IL-17 antibody that binds to an epitope of an IL-17 homodimer having two mature IL-17 protein chains, the epitope comprising Leu74, Tyr85, His86, Met87, Asn88_{;} Val124, Thr125, Pro126, Ile127, Val128, His129 on one chain and Tyr43, Tyr44, Arg46, Ala79, Asp80 on the other chain, wherein the IL-17 antibody has a K_{D} of about 100-200 pM, and wherein the IL-17 antibody has an in vivo half-life of about 23 to about 35 days; and
e) are IL-17 antibody comprising:
i) an immunoglobulin heavy chain variable domain (V_{H}) comprising the amino acid sequence set forth as SEQ ID NO:8;
ii) ara immunoglobulin light chain variable domain (V_{L}) comprising the amino acid sequence set forth as SEQ ID NO:10;
iii) immunoglobulin V_{H} domain comprising the amino acid sequence set forth as SEQ ID NO:8 and an immunoglobulin V_{L} domain comprising the amino acid sequence set forth as SEQ ID NO:10;
iv) an immunoglobulin V_{H} domain comprising the hypervariable regions set forth as SEQ ID NO:1, SEQ ID NO:2, and SEQ ID NO:3;
v) an immunoglobulin V_{L} domain comprising the hypervariable regions set forth as SEQ ID NO:4, SEQ ID NO:5 and SEQ ID NO:6;
vi) are immunoglobulin V_{H} domain comprising the hypervariable regions set forth as SEQ ID NO:11, SEQ ID NO:12 and SEQ ID NO:13;
vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
viii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ NO:4, SEQ ID NO:5 and SEQ ID NO:6.

58. The method according to claim 57, wherein the IL-17 antagonist is secukinumab.
